(19) United States
(12) Patent Application Publication
(10) Pub. No.: US 2002/0048584 A1
(43) Pub. Date: Apr. 25, 2002

(54) Method of treating residual HIV-I disease

(76) Inventor: Roger J. Pomerantz, Chalfont, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/861,751

(22) Filed: May 21, 2001

Related U.S. Application Data
(60) Provisional application No. 60/205,667, filed on May 19, 2000.

(51) Int. Cl. C12Q 1/70; A61K 45/00; A61K 39/395

(52) U.S. Cl. 424/153.1; 435/5

(57) ABSTRACT

The current therapeutic approach to treating patients with HIV-1 infection involves the use of one or more highly active antiretroviral therapeutics (HAART). While efficacious, this approach does not address the existance of latently infected cells. Such latently infected cells can be reactivated, resulting in the expression of infectious virus and reinitiation of the disease process. The present invention relates to a novel and highly efficacious approach to eradication of HIV-1. Patients that are treated with HAART are then treated with an intensification regimen wherein hydroxyurea and didanosine (ddI) are given to inhibit any residual viral replication. The therapeutic regimen is continued with the addition of compounds, such as OKT3 and IL-2, that activate latently infected cells, thereby stimulating the replication of any proviruses. These re-activated viruses are subsequently inhibited by the HAART and hydroxyurea/ddI therapeutics. Thus, the present invention provides a method of treating HIV-1 to eradicate any low-level viral replication and latently infected cells, thereby eliminating residual HIV-1 disease.

### CROSS REFERENCE TO RELATED APPLICATIONS

This application claims priority under 35 U.S.C.§119 based upon U.S. Provisional Application No. 60/205,667 filed May 19, 2000.

### FIELD OF THE INVENTION

The present invention relates to the fields of virology and infectious diseases, and to a method of treating a patient with an HIV-1 infection and, more particularly, to the use of hydroxurea and didanosine (ddI) as an intensification therapy and OKT3 and IL-2 as a stimulation therapy, thereby eliminating any residual HIV-1 infected cellular resevoirs.

### BACKGROUND OF THE INVENTION

HIV-1 Reservoirs during Effective HAART

Treatment of human immuodeficiency virus type I (HIV-1) in the developed world has undergone remarkable changes over the last several years. With the advent of highly active antiretroviral therapy (HAART), which usually includes a combination of reverse transcriptase and protease inhibitors, a significant majority of patients in many cohorts of HIV-1-infected-individuals may obtain clinically undetectable levels of viral RNA in the peripheral blood plasma. Correlated with these alterations in HIV-1 expression in the peripheral blood are dramatic changes in mortality and morbidity. As such, effective HAART has led to many HIV-1 infections in vivo being converted to chronic or at least subacute disease states.

Along with this advent of viable and robust therapy, there have been dramatic changes in the understanding of the in vivo pathogenesis of this human lentiviral disease. The critical papers by Drs. D. Ho and G. Shaw led to a clear understanding of HIV-1 replication within the human host. These studies demonstrated that HIV-1 replication takes place at a remarkable rate in the peripheral bloodstream and lymphoid tissue of infected individuals. Huge numbers of virion particles are produced on a daily basis from productively-infected T-lymphocytes. The turnover of virions yields an in vivo half-life of less than several hours and the productively-infected CD4+ T-lymphocytes appear to turnover at a rate of approximately 1½ days. As such, a “viral brush-fire” continues unabated in most infected individuals prior to treatment.

Productively-infected CD4+ T-lymphocytes account for greater than 99% of infected cells in the peripheral blood and lymphoid tissue. As such, effective HAART can virtually “turn-off” the vast majority of viral production and on-going CD4+ T-cell depletion. When HAART leads to viral RNA loads in the peripheral blood of less than 400 copies/ml, or more appropriately, less than 50 copies/ml, this correlates with the dramatic inhibition of virtually all viral replication in the infected-host. Nevertheless, data have also been obtained by the laboratories of Drs. D. Richman, R. Silicano and A. Fauci which demonstrate that resting CD4+ T-lymphocytes can maintain proviral species in these individuals. Moreover, replication-competent virus has been generated from these proviral-harboring cells in the peripheral blood and lymphoid tissue of patients on effective HAART with undetectable viral RNA in the peripheral blood plasma.

Thus, these reservoirs lead to yet another obstacle in the path of developing eradication protocols for HIV-1 in vivo infections. Understanding the molecular pathogenesis and cellular reservoir sites for these low but critical levels of proviral-harboring cells is now one of the key issues in HIV-1 research.

In Vivo Pathogenesis

Molecular aspects of HIV-1 pathogenesis and transmission in vivo have been analyzed in detail for over fifteen years. Significant understanding of these aspects of HIV-1 infection has developed over this time-period. Importantly, many successes in anti-EIV-1 treatments have been rationally designed, based on a clear understanding of the viral life-cycle and HIV-1 pathogenesis (O'Brien, W., Pomerantz, R. J., Viral Pathogenesis. New York: Raven Press, 1997: 813-837).

HIV-1 replicates, in most infected individuals, at high levels throughout infection, including the clinical quiescent phase. Levels of this active viral replication directly correlate with disease progression and survival (Ho, D. D., et al., Nature 373: 123-126, 1995; Mellors, J., et al., Science 272: 1167-1170, 1996; Piatak, M., et al., Science 259: 1749-1754, 1993). Combination therapeutics for HIV-1 (supra) lead to dramatic alterations in viral replication in vivo. With HAART's ability to inhibit HIV-1 viral load (virion-associated RNA) to undetectable levels in the blood plasma and genital fluids of many infected individuals, one can now formally analyze whether proviral-harboring genital tract cells can transmit HIV1 sexually, without cell-free virions. Further, mechanisms of proviral persistence and “cryptic” viral replication can now be addressed without the “noise” of active virally-producing cells and high levels of cell-free virions (Wong, J. K., et al., Science 278: 1291-1295, 1997; Finzi, D., et al., Science 278: 1295-1300, 1997; Chun T. W., et al., Proc. Natl. Acad. Sci. 94: 13193-13197, 1997; Zhang, H. et al., N. Engl. J. Med., 339: 1803-1809, 1998). Thus, by analyzing virological parameters, especially the presence or absence of 2-LTR circular DNA (an end-stage form of retroviral DNA) in genital secretions and mechanisms of transmission of HIV-1 in patients on HAART with undetectable virus in blood plasma, clinically-relevant, immediately-critical questions are answered, as well as issues involving future attempts at in vivo lentiviral eradication.

HIV-1 Persistence

Interest in retroviral latency, or more precisely persistence, preceded the AIDS epidemic. Nevertheless, the understanding of restricted retroviral replication in vivo has significantly increased in recent years, utilizing HIV-1 as a model (O'Brien, W., Pomerantz, R. J., Viral Pathogenesis. New York: Raven Press, 1997:813-837). It is critical at the outset to define the concepts of cellular persistence or latency in vivo for retroviruses. Many studies have demonstrated that latency for HIV-1, as defined as no viral expression in an untreated infected individual, does not exist at the organismal level in any stage the of disease (Embretson, J., et al., Nature 362: 357-362, 1993; Pantaleo, G., et al., Nature 362: 355-358, 1993; Piatak, M., et al., Science 259: 1749-1754, 1993). Yet, in the great majority of HIV-1-infected-individuals, some cultivable virus may be recovered at all stages of disease (Embretson, J., et al., Nature 362: 357-362, 1993; Pantaleo, G., et al., Nature 362: 355-358, 1993; Piatak, M., et al., Science 259: 1749-1754, 1993; Pantaleo, G., et al., Nature 362: 355-358, 1993; Piatak, M., et al., Science 259: 1749-1754, 1993). Data have been reported, using a wide variety of techniques, indicating that cells exist in the infected individual that harbor the HIV-1 provirus but express little or no viral RNA and produce few or no virions (Embretson, J., et al., Nature 362: 357-362, 1993; Patterson B., et al., Science 260: 976-979, 1993; Peng H., et al., Virology 206: 16-27, 1995). As such, “latency” or persistence at a cellular level may exist in vivo, and the numbers of latently-infected cells may vary based on the stage of disease.

The HIV-1 life-cycle contains many possible sites for restricted replication (O'Brien, W., Pomerantz, R. J., Viral Pathogenesis. New York: Raven Press, 1997: 813-837). As HIV-1 infects, in vivo, both CD4+ T-lymphocytes and monocyte/macrophages (Schnittman, S. M., et al., Science 245: 305-308, 1989; Koenig, S. et al., Science 233: 1089-1093, 1986), the virus may maintain cellular latency by different mechanisms in different cell-types. The major, but not sole, cellular reservoir for HIV-1 in the peripheral bloodstream is the CD4+ T-lymphocyte. Monocytic cells, which are the main viral reservoir in most solid tissues, thus may be fundamentally different in their replication of HIV-1 (Schnittman, S.M., et al., Science 245: 305-308, 1989; Koenig, S. et al., Science 233: 1089-1093, 1986).

Stages of HIV-1 Cellular Latency

Various states of HIV-1 cellular latency prior to integration may exist in cell cultures and in vivo. Studies have demonstrated that T-lymphocytes not activated by mitogens do not allow productive replication of HIV-1 in cell culture. In one report, incomplete HIV-1 reverse transcription occurred, as measured by the polymerse chain reaction (PCR), leading to unstable partially reverse-transcribed HIV-1 DNA intermediates (Zack, J., et al., J. Virol. 61: 213-333, 1990). The completion of reverse transcription and integration of these intermediates could occur after infected cells are stimulated with mitogen. It was suggested that viral DNA intermediates may survive in resting T-lymphocytes, in vivo, and provide a form of pre-integration latent infection. Further data suggest that the efficiency of the completion of viral DNA production, from partial reverse transcripts, is rather poor in some cell-types (Zack, J. et al., J. Virol. 66: 1717-1726, 1992).

In a second report, evidence was provided that unstimulated T-lymphocytes in cell culture can fully reverse-transcribe HIV-1-specific RNA but the HIV-1 DNA produced in this process does not integrate into the host genome, as the pre-integration complex does not undergo cytoplasmic to nuclear transport. Only after cellular stimulation by phytohemagglutinin (PHA) is the HIV-1 double-stranded DNA able to integrate and productively express progeny virions (Bukrinsky, M., et al., Science 254: 423-427, 1991; Stevenson, M., et al., EMBO J. 9: 1551-1560). Unintegrated HIV-1 DNA species were demonstrated in peripheral blood lymphocytes of certain HIV-1-infected individuals (Bukrinsky, M., et al., Science 254: 423-427, 1991). Stimulation of these cells with mitogens in cell culture led to integration of the viral DNA. It has been proposed that unintegrated, linear HIV1 DNA structures may function as a reservoir of latent HIV-1 infection in resting T-lymphocytes in vivo (Bukrinsky, M., et al., Science 254: 423-427, 1991).

Model Systems to Study HIV-1 Latency

Cell lines have been selected from the survivors of lytic HIV-1 infections that maintain HIV-1 in the restricted state and constitutively produce very low levels of the virus (Pomerantz, R. J., et al., Cell 61: 1271-1276; Butera, S. T., et al., J. Virol. 68: 2726-2730, 1994; Michael, N. L., et al., J. Virol. 69: 2977-2988, 1995). These cell lines can be stimulated to increase HIV-1 expression with a variety of exogenous compounds. Most of these compounds appear to act via activation of nuclear factor-κB (NF-κB). Two HIV-1 latently-infected cell lines have been extensively characterized, the Ul monocytic and the ACH-2 T-lymphocytic lines. These cell-lines have been used as model systems to explore certain aspects HIV-1 post-integration latency in cell culture. In the baseline unstimulated state, these cells express multiply-spliced HIV-1-specific RNA, as compared to productively-infected cells in which all three HIV1 RNA species are expressed in nearly equivalent amounts. This RNA expression pattern undergoes a switch to mainly synthesis of unspliced transcripts upon stimulation of these cells. Cells expressing mainly or solely multiply-spliced viral RNA have also been demonstrated in initial studies of viral persistence (Pomerantz, R. J., et al., Cell 61: 1271-1276; Butera, S. T., et al., J. Virol. 68: 2726-2730, 1994; Michael, N. L., et al., J. Virol. 69: 2977-2988, 1995).

Molecular Mechanism of Restricted Replication

Restricted replication of HIV-1 in vivo could be produced through a wide variety of molecular mechanisms (supra). For instance, cells might contain proviral DNA but lack any viral RNA expression (Spina, C. A., et al., J. Virol. 69: 2977-2988, 1995). Partially defective viral genomes, demonstrated in certain cells in vivo, might also lead to latent cellular infections (Patterson, B. et al., Science 260: 976-979, 1993; Sanchez, G., et al., J. Virol. 71: 2233-2240, 1997; Wei X., et al., Nature 3: 117-122, 1995). Importantly, studies have demonstrated that the number of cells in the peripheral blood of HIV-1 infected persons that harbor the proviral genome is much higher than the numbers of cells that express high levels of HIV-1-specific RNA (Embretson, J., et al., Nature 362: 357-362).

HIV-1 Persistance is Sensitive to Cell Location

Cellular HIV-1 persistence may have a further level of control, in which the quantity of HIV-1 production within a cell may not only be based on the cell-type but also may be affected by the location, within the body, of a particular cell. The control of HIV-1 proviral latency in monocyte/macrophages may significantly differ, based on whether the monocytic cell is in the CNS, the bone marrow, or the liver. Additionally, the CD4+ T-lymphocyte, infected with HIV-1, may differ in its level of viral expression and its cellular activation parameters depending on whether it is found in the peripheral blood or a lymph node. Thus, cellular persistence of HIV-1 may be based on multiple levels of complexity tied to the molecular form of latent infection, the cell-type and the location of the infected cell within an HIV-1-infected-individual (O'Brien, W., Pomerantz, R. J., Viral Pathogenesis. New York: Raven Press, 1997: 813-837).

It has been demonstrated that HIV-1 replicates at a rapid rate in infected individuals, with a virion T_{½} of less than 6 hours (Perelson, A. S., et al., Nature 387:188, 1997). The vast majority of this viral replication (up to 99%) occurs in activated and productively-infected CD4+ T-lymphocytes in the peripheral blood and lymphoid tissue (FIG. 1). Nevertheless, using viral decay characteristics in patients initially treated with HAART, second and third phase decay of plasma viremia occurs, secondary to long-lived infected cells (e.g., tissue-bound macrophages) and latently-infected T-lymphocytes (FIG. 1) (Perelson, A. S., et al., Nature 387:188, 1997). Thus, HIV-1-infected-individuals will probably require standard HAART for prolonged periods of time (greater than three years), if one is going to consider viral eradication. Viral eradication may also be greatly hindered by infection in other in vivo tissue compartments and sanctuary sites. Tissues that maintain blood:tissue barriers, secondary to microvascular endothelial tight junctions, may hinder penetration of anti-retroviral agents. These compartments would potentially include the testes, the central nervous system (CNS), and the retina.

HAART Does Not Eliminate Cells with Latent Virus

Persistently-infected, non-activated CD4+ T-lymphocytes have been demonstrated in the peripheral blood of HV-1-infected-individuals (Chun, T. W. et al., Nature 387: 183-188, 1997). Of importance, these proviral-harboring cells have now been demonstrated in infected individuals treated with HAART who have undetectable viral RNA in blood plasma (i.e., below 50 copies per milliliter). It has also been shown that replication-competent viruses can be recovered from these proviral-positive cells (Wong, J. K., et al Science 278: 1291-1295; Finzi, D., Science 278: 1295-1300, 1997; Chun, T. W., et al., Proc. Natl. Acad. Sci. 94: 13193-13197, 1997). These proviral-harboring T-lymphocytes develop very soon after initial HIV-1 seroconversion (Chun, T. W., et al., Proc. Natl. Acad. Sci. 95: 8869-8873, 1998). One study suggested, indirectly by quantitating total HIV-1 DNA and subtracting integrated HIV-1 DNA, that some low level viral replication may still take place in certain of these cells indicating unintegrated viral DNA may be present. (Chun, T. W., et al., Proc. Natl. Acad. Sci. 94: 13193-13197, 1997). Further studies are necessary to analyze the potential for “cryptic” viral replication in these cell populations. Of note, very low-level viral transcription may take place in lymph nodes of selected patients on “effective” HAART (Cavert, W., et al., Science 276: 960-964, 1997).

The replication-competent viruses, isolated from proviral-harboring T-lymphocytes in patients on HAART with undetectable viral RNA in blood plasma have been demonstrated to have little or no anti-retroviral resistance mutations (Wong, J. K., et al Science 278: 1291-1295). As resistance mutations in the reverse transcriptase (RT) and protease (PR) genes of HIV-1 are correlated with on-going viral replication (Gunthard, H. F., et al., J. Virol. 72: 2422-2428, 1998), this suggests that shortly after primary seroconversion these viral strains may represent “archival” species. These data also suggest that, although defective proviruses accumulate in CD4+ T-lymphocytes in vivo (i.e., “viral graveyards”) (Sanchez, G., et al., J. Virol. 71: 2233-2240, 1997), there still exits replication-competent proviruses in persistently-infected cells. These persistently-infected cells hinder attempts at eradication by re-seeding the body with virus if HAART is discontinued in yet to be determined time-periods following the primary infection. Analysis of in vivo decay times of these persistently-infected cells is now of critical importance.

Persistance and Transmission

Sanctuary sites

Long-standing interests in molecular aspects of viral persistence and transmission exist. Initial studies with Dr. Martin Hirsch were some of the first to demonstrate HIV-1 infection in the cervix of infected women (Pomerantz, R. J., et al., Ann. Int. Med. 108: 321-327, 1988). In addition, previous studies demonstrated HIV-1 infection of the human retina in vivo. This area could act as a potential sanctuary site, as it is contained by a blood:retina barrier, analogous to the central nervous system central nervous system (CNS) (Pomerantz, R. J. et al., N. Engl. J. Med. 317: 1643-1647, 1987).

To investigate the impact of HAART on HIV-1 replication in the male genital tract, peripheral blood and semen fluids were simultaneously collected from seven HIV-1-infected men receiving triple anti-retroviral therapy for significant time-periods and having repeatedly undetectable viral RNA levels in the peripheral blood. Both the viral RNA levels in the blood plasma and seminal fluid of these patients were demonstrated to be below 50 copies/ml. These results suggest that the HAART utilized in these individuals could not only potently inhibit viral replication in the bloodstream but also in the seminal fluids. Importantly, cell-associated viral DNA was detected in all of the patients' peripheral blood mononuclear cell (PBMC) samples (Zhang, H. et al., N. Engl. J. Med. 339: 1803-1809, 1998).

Of note, cell-associated proviral DNA was also detected in the seminal cells from four of the above patients. For two patients, the seminal cell- and PBMC-associated HIV-1 proviral DNA was examined at two different time-points (separated by two to three months). There was only modest variation in the seminal cell-associated proviral DNA in these patients. Thus, the PBMC and seminal proviral levels differed relatively little in these two patients over a few month time-period, suggesting the relative stability of these HIV-1 proviral species over at least the short-term (Finzi, D., et al., Science 278: 1295-1300, 1997; Chun, T. W., et al., Nature 387: 183-188, 1997).

Seminal Cells Harber HIV-1 DNA

As cell-free HIV-1 in the seminal plasma was very low or absent in these patients, HIV-1 DNA-positive seminal cells were examined to determine whether they could harbor replication-competent virus. HIV-1 replication was found in seminal cell samples from two of the seven individuals. The CD8+ T-lymphocytes depleted from the co-culture system are very important for the primary isolation of viruses from these PBMC (supra) and seminal cells (Piatak M., et al., Science 259: 1749-1754, 1993; Gulick, R. M. et al., N. Engl. J. Med. 337: 734-739, 1997; Palella, F. J., et al., N. Engl. J. Med. 338: 853-860, 1998; Hammer, S. M., et al., N. Engl. J. Med. 337: 725-733, 1997).

To investigate whether the replication-competent viruses recovered from seminal cells were capable of being sexually transmitted, the sequences in the V₃ loop of the gp120 envelope (env) region and the replication phenotype of the recovered viruses were examined. According to the net amino acid charges in the V₃ loop and the growth pattern of the viruses in cell culture (Fouchier, R. A. M., et al., J. Virol. 66: 3183-3187, 1992), seminal cell-derived virus isolated from one patient was typical macrophage-tropic strain, while the virus isolated from another patient D appeared to be a dual-tropic strain. Growth of virus in MT-2 T-lymphocytes denotes a T-cell line tropic HIV-1 isolate, utilizing primarily the CXCR4 chemokine co-receptor (X4 strain), while growth in primary human macrophages demonstrates a viral isolate which usually utilizes primarily the CCR5 chemokine co-receptor (R5 strain) (Littman, D. R., Cell 93: 667-680, 1998). As most HIV-1 strains detected during sexual transmission are macrophage- or dual-tropic (utilizing CCR5 or both CCR5 and CXCR4, respectively) (Zhu, T. et al., Science 261: 1179-1181, 1993; Zhang, L. Q., et al., J. Virol. 67: 3345-3356, 1993; Zhu, T., et al., J. Virol. 79: 3098-3107, 1996; van't Wout, A., et al., J. Clin. Invest. 94: 2060-2067, 1994), these results indicate that the replication-competent viruses isolated from the seminal cells are potentially capable of initiating primary infection and transmission to a sexual partner, even though these patients were on HAART and viral RNA in the blood plasma remains undetectable. The sequences of the V₃ loops also indicated that the recoverable, replication-competent viruses from seminal cells and PBMCs are most likely derived from the proviral DNA in these cells.

It has been reported that reverse transcriptase inhibitor-resistant, as well as protease inhibitor-resistant, HIV-1 can be transmitted between sexual partners (Veenstra, J. R., et al., Clin. Infect. Dis. 21: 556-560, 1995; Ippolito, G., et al., JAMA 272: 433-434, 1994; Angarano, G., et al., AIDS 8: 1013-1014, 1994). To determine the drug sensitivity of the replication-competent HIV-1 viruses and proviral DNA from the seminal cells and PBMCs of the aforementioned two patients, RT and PR regions were sequenced. A single drug resistance mutation was found in the PR gene (L10I) of recovered virus from the PBMCs and PBMC proviral DNA of one patient. No drug resistance mutations were detected in either the recovered replication-competent virus from the seminal cells or seminal cell-associated proviral DNA in either patient (Zhang, H. et al., N. Engl. J. Med. 339: 1803-1809, 1998).

The existance of a proviral reservoir of replication-competent virus in the seminal cells of selected men on HAART therapy (supra), in whom HIV-1 RNA was undetectable in both the peripheral blood plasma and seminal fluid, represents a critical reservoir site which has significant clinical implications. Firstly, this reservoir of persistently-infected cells in the genital tract will allow sexual transmission of the HIV-1, via a cell-associated route, even if the patients lack free HIV-1 virions in the blood plasma and seminal fluid. Furthermore, this represents yet another in vivo reservoir sanctuary site which may reinfect the peripheral bloodstream and lymphoid tissue if HAART is discontinued, thereby hindering attempts to eradicate the virus. Of note, a proviral reservoir in the peripheral blood, and possibly other sites, appears to be present very soon after primary HIV-1 seroconversion (Piatak, M., et al., Science 259: 1749-1754, 1993).

An Initial HIV-1 Eradication Protocol

Effective combination therapy referred to as HAART (supra) has changed the HIV-1 epidemic, at least in the developed world (Gulick, R. M., et al., N. Engl. J. Med. 337: 734-739, 1997; Palella, F. J., et al., N. Engl. J. Med. 338: 853-860, 1998; Hammer, S. M., et al., N. Engl. J. Med. 337: 725-733, 1997). Viral RNA in the peripheral blood of a majority of individuals on HAART may have HIV-1 inhibited to undetectable levels. Nevertheless, recent data from several groups have demonstrated a proviral reservoir in resting CD4+ T-lymphocytes of the peripheral blood and lymphoid tissue of patients treated with HAART who lack detectable viral RNA in the peripheral blood plasma (supra) (Chun, T. W., et al., Proc. Natl. Acad. Sci. 95: 8869-8873, 1998; Wong J. K., et al., Science 278: 1291-1295, 1997; Finzi D., et al., Science 278: 1295-1300; Chun, T. W., et al., Proc. Natl. Acad. Sci. 94: 12139-12197). This reservoir may be critical in the future pathogenesis of the disease (O'Brien, W., Pomerantz, R. J., Viral Pathgenesis. New York: Raven Press, 1997: 813-837) and in altering the ability to seek eradication utilizing standard HAART.

Long-lived Cells Continue to Shed Virus

The replication-competent HIV1 isolated from the seminal cells of HIV-1-infected men receiving HAART serves as a reservoir in the male genital tract for HIV-1 replication in vivo (supra). (Pantaleo, G. C. et al., Nature 362: 355-358, 1993). This phenomenon could be due to the decay at a very slow rate of certain cells harboring HIV-1 proviral DNA in the male genital tract. Theoretically, if no anti-retroviral drug resistant viral mutants develop during HAART, re-infection of cells by HIV-1 would not continue in the microenvironments, in vivo, where the anti-viral agents reach inhibitory levels. As re-infection is consistently inhibited, the decay of HIV-1-infected cells continues. However, if the life-span of certain cells harboring intact HIV-1 proviral DNA is long, viruses wil be shed in select circumstances. Furthermore, low-level replication of HIV-1 could still occur, thereby re-infecting cells in the male genital tract tissues and/or fluids. As the blood:testes barrier may prevent anti-viral agents from effectively entering testicular tissue, a partial “drug sanctuary” could exist.

It remains to be clarified which specific cells in the genital tract of HIV-1-infected men receiving HAART harbor replication-competent HIV-1. Controvery existsl regarding which cells in the male genital tract are targets for HIV-1 infection in untreated, infected men. Macrophages and T-lymphocytes in the seminal secretions are primary target cells for HIV1 infection (Quayle, A. J., et al., J. Infect. Dis. 176: 960-968, 1997). There is also some evidence to support at least low levels of HIV1 proviral DNA in the self-renewing spermatogonia and their progeny, although these may be defective or incomplete proviral sequences (Baccetti, B., et al., J. Cell Biol. 127: 903-914, 1994; Nuovo, G. J. et al., Amer. J. Path. 44: 1142-1148, 1994; Bagasra, O., et al., AIDS 8: 1669-1974, 1994). As no CD4 molecules were detected on the surfaces of spermatogonia (Gil, T., at al., Human Reprod. 10: 2923-2927, 1995), it remains a question of how HIV-1 may enter germ cells. However, a group of α- and β-chemokine receptors have been identified as the co-receptors for HIV-1, and CD4-independent infection has been demonstrated in certain cell lines with several primate lentiviruses (Edinger, A. L., et al., Proc. Natl. Acad. Sci. 94: 14742-14747, 1997; Endres, M. J., et al., Cell 87: 745-756, 1996). Consequently, it would be interesting to examine whether chemokine receptors are expressed on select cells in the male genital tract. Such cells include, but are not limited to, epithelial cells, self-renewing spermatogonia and their progeny (Table 1).

TABLE 1
Potential sites of HIV in the male and female genital tracts
Male-to-male and male-to female transmission

1. Cell-free virions in seminal plasma
2. Seminal mononuclear cells

a. Cell-associated virions
b. Productively or persistently infected cells

3. Germ cells

a. Virions bound to sperm
b. Proviral HIV DNA

Female-to-male transmission

1. Cell-free virions in cervicovaginal secretions
2. Cervicovaginal macrophages and other cell types

a. Cell-associated virions
b. Productively or persistently infected cells

In the case of fully successful or effective HAART, re-infection of PBMC and seminal cells should not occur or be at very low levels. Resting CD4+ T-lymphocytes from the local lymphoid tissue, which may have relatively long life-spans, could be reservoirs harboring HIV-1. As some of the sequences of HIV-1 from the seminal cells are significantly different from those obtained from peripheral blood lymphocytes (PBL), certain of the infected seminal T-lymphocytes may not have trafficked from the peripheral bloodstream directly. These cells could be stimulated in situ by cytokines or allogenic cells in the in vitro co-culture (infra) system to generate small quantities of HIV-1 virions, which are then recoverable by co-culture assay (Chun, T. W., et al., J. Exp. Med. 188: 83-91, 1998). Conversely, the life-span of macrophages, or any monocyte-derived cells, in the local tissues remains to be clarified. These cells could also harbor HIV-1 provirus if their in vivo decay rate is slow enough. Finally, in the testicular tissue or semen, self-renewing spermatogonia may harbor HIV-1 DNA and shed HIV-1 virions under specific conditions (Nuovo, G. J., et al., Amer. J. Path. 44: 1142-1148, 1994).

Seminal Cells—Vehicles for Sexual Transmission of HIV-1

The seminal cells harboring proviral DNA could be vehicles for sexual transmission of HIV-1 from infected men to sexual partners. These infected cells could directly contact the target cells in the mucosa of sexual partners, yielding transmission of HIV-1 (Phillips, D. M., AIDS 8: 719-731, 1994). The HIV-1 virions produced in infected men receiving HAART are infectious since the anti-viral agents are diluted to non-functional levels in the semen, thereby resulting in the transfer of virus to the sexual partner via the seminal cells. These replication-competent viruses remain anti-retroviral drug-sensitive. Genotypic and phenotypic analyses indicate that the replication-competent viruses recovered from the seminal cells are macrophage- or dual-tropic, implying they have the potential to initiate and establish primary infection in sexual partners (Zhu T., et al., Science 261: 1179-1181, 1993; Zhang L. Q., et al., J. Virol. 67: 3345-3356, 1993; Zhu T. et al., J. Virol. 79: 3098-3107, 1996; van't Wout, A., et al., J. Clin. Invest. 94: 2060-2067, 1994). Further, the HIV-1 proviral DNA in seminal cells, from which these viruses are likely derived, may represent “archival” or “fossil” viral sequences from cycles of replication early after primary infection. This may be based on seminal cells infected at the start of anti-retroviral treatment, but could also be due to viral replication occurring at low levels in a relative “drug sanctuary” region. Therefore, the HIV-1 strains with potential to be sexually-transmitted by infected-men on HAART in whom blood and seminal viral RNA levels are maintained at undetectable levels are likely to be anti-retroviral sensitive in many cases. This may differ from those patients on combination anti-retroviral therapy but with on-going and detectable viral replication in vivo (Hecht, F. M. et al., N. Engl. J. Med. 339: 307-312, 1998).

It has been demonstrated that replication-competent viruses can be recovered from seminal cells of HIV-1-infected men receiving long-term HAART. These patients, while following the HAART regimens and with viral loads in the blood plasma remaining below detectable levels, still maintain a proviral reservoir in their genital tract and are a source for transmission of HIV-1 to their sexual partners. Thus, these findings have significant public health importance. Furthermore, these infected seminal cells hinder attempts at viral eradication in vivo.

Viral Sanctuaries Allow for Incomplete Suppression of HIV-1

Recent studies have demonstrated higher seminal viral load in patients in the later stages of HIV-1 disease, which decreases with anti-retroviral therapy (Hamed, K. A., et al., J. Infect. Dis. 167: 798-802, 1993; Gupta, P., et al., J. Virol. 71: 6271-6275, 1997). Nevertheless, although these previous studies have analyzed HIV-1 in seminal secretions during anti-retroviral therapy (Hamed, K. A., et al., J. Infect. Dis. 167: 798-802, 1993; Gupta, P., et al., J. Virol. 71: 6271-6275, 1997; Vemazza, P. L., et al., AIDS 11: 1249-1254, 1997; Anderson, D. J., et al., JAMA 267: 2769-2274, 1992; Xu, C., et al., J. Infect. Dis. 176: 941-947, 1997; Vernazza, P. L., et al., AIDS 11: 987-993, 1997), they did not evaluate fully suppressive HAART in which viral plasma RNA levels were undetectable. A very recent and instructive study demonstrated different resistance mutations in HIV-1 RNA from seminal fluid, as compared to blood plasma (Eron, J. J., et al., AIDS 12: F181-F189, 1998). Furthermore, in certain patients who responded poorly (i.e., virologically) to anti-retroviral therapy, viral levels remained more elevated in seminal fluid versus blood plasma. This implies incomplete suppression, secondary to limited drug penetration, into the male genital tract (Enting, R. H., et al., AIDS 12: 1941-1955, 1998). These data further support a viral sanctuary site in the seminal fluid.

The combined studies in retroviral transmission and persistence (e.g., sanctuary sites, persistence, and cryptic replication) are of a critical nature. Therefore, discontinuing anti-viral therapy in infected-individuals in whom viral RNA levels in the peripheral blood plasma have been reduced to undetectable levels is of concern. The present invention addresses these issues by providing therapeutic approaches towards altering potential transmission to sexual partners and ablating the potential for future viral replication.

### ABBREVIATIONS

“PBMC” means “peripheral blood mononuclear cells”

“HAART” means “highly active antiretroviral therapy”

“ddI” means “didanosine”

“PBL” means “peripheral blood lymphocytes”

“PBS” means “phosphate buffered saline”

“bid” means “twice daily”

“po” means “by mouth”

“qday” means “every day”

### SUMMARY OF THE INVENTION

The present invention provides a method for treating a patient with HIV-1 while the patient is receiving HAART. A patient is selected for therapy and administered an intensification therapeutic(s), the therapeutic(s) are administered in an amount sufficient to block reverse transcriptase. The patients cells and plasma are monitored for decreases in proviral sequences and replication-competent viruses. They are then administered a compound to activate latently-infected cells, the compound is administered in an amount sufficient to activate expression of the latent virus. A second compound is administered to further activate the latently infected cells, the second compound being administered in an amount sufficient to further activate expression of the latent virus. The patients cells and plasma are analyzed for the proviral sequences and replication-competent viruses. This method is efficacious in eradicating HIV-1.

In one embodiment the intensification therapeutic(s) are hydroxyurea and ddI. In another embodiment the compound to activate latently infected cells is OKT3. In a further embodiment the second compound to further activate latently infected cells is IL-2.

In one embodiment activation of the expression of the latent virus is by depleting proviral resevoirs. In another embodiment the cells are within a sanctuary site. In another embodiment the sanctuary site is at least one of a gential tract, a central nervous system, or a retina.

### DESCRIPTION OF THE DRAWINGS

FIG. 1. Viral infectivity in T-lymphocytes.

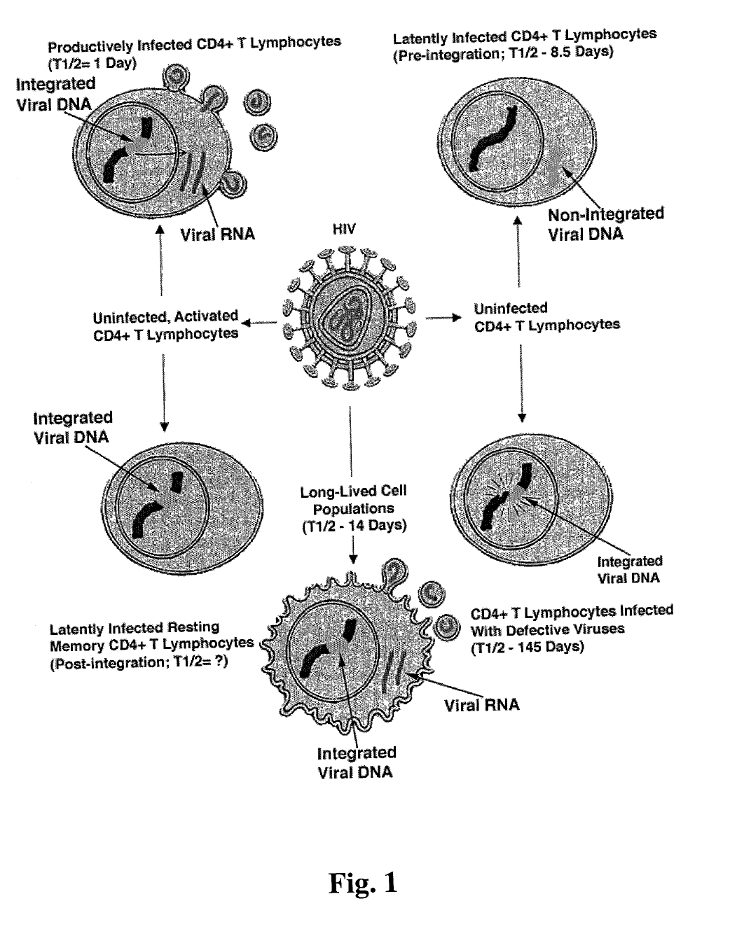

FIG. 2. Residual HIV-1 during suppressive HAART.

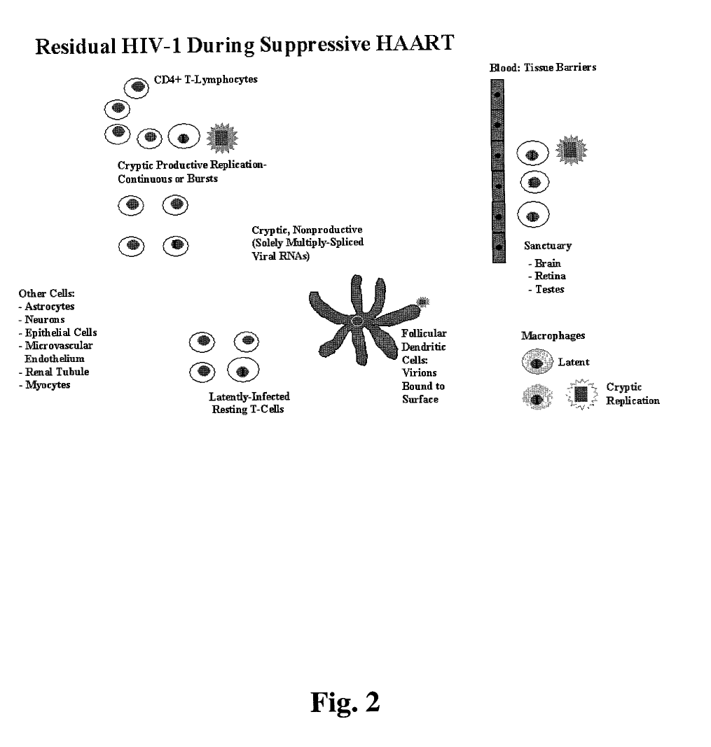

FIG. 3. Residual HIV-1 Eradication (RHIDE) Protocol

FIG. 4. Intensification Protocol Outline

FIG. 5. Residual HIV-1 Disease Eradication Protocol (RHIDE)

FIG. 6. Residual HIV-1 Disease Eradication Protocol Study—Patient #28

FIG. 7. Residual HIV-1 Disease Eradication Study on Patient #28 after all anti-retrovirals have been stopped.

### DESCRIPTION OF THE INVENTION

HIV-1 reservoir sites during effective HAART (supra) are of critical importance in the potential for developing techniques to eradicate HIV-1 infection in the human host. Clearly, HIV-1 latency or persistence must be analyzed on a variety of levels. The cell-types that harbor latent proviral species, including resting CD4+ T-lymphocytes, certain monocyte/macrophage populations and possibly other cell-types will require investigation. In addition to cell types, the stage in the viral life-cycle is critical for determining the aggressiveness of the treatment regimen. The detection of HIV-1 2-LTR DNA moieties are a direct correlate of new viral infections of cells, thus analyzing for the presence of 2-LTR will allow low levels of covert lentiviral replication to be detected. (Domadula, G., et al., Journal of Infectious Diseases in press, incorporated herein by reference). In addition, possible sanctuary sites behind blood:tissue barriers (such as the central nervous system, testes and retina) are also critical areas of investigation.

It is important to further understand the molecular mechanisms involved with latently-infected cells in patients on effective HAART. Whether pre-integration reverse transcripts are a major species in these cells, or whether most are integrated proviruses, require further analyses, rather than relying on previous studies in patients who are not on effective HAART. Additionally, the pre-integration species may be either partial or full reverse transcripts. It is possible that “cryptic” replication may also be taking place in certain cells in patients on effective HAART. This replication of HIV-1 does not lead to detectable levels in the peripheral blood plasma. This replication could be both low and constant or in bursts.

The treatment options in attempts to rid the body of cells infected with HIV-1, which can produce replication-competent virus in patients on effective HAART, should be based on a rational design secondary to further understanding of the in vivo molecular pathogenesis.

Hydroxurea/ddI Regimen

The present invention further relates to the use of hydroxyurea as an intensification therapeutic to inhibit viral replication. Hydroxyurea has been used as a modality in several HAART regimens, especially combined with didanosine (ddI), with which it has synergistic anti-HIV-1 effects (Giacca, M., et al., J. Infect. Dis. 174: 204-209, 1996; Rutschmann, O. T., et al., AIDS 12: F71-F77, 1998). Hydroxyurea is a novel approach to anti-retroviral therapy as it selectively inhibits ribonucleotide reductase, acting as a scavenger for tyrosyl free radical, essential for enzyme activity. Inhibition of cellular ribonucleotide reductase leads to a dramatic decrease in intracellular dNTP pools. As such, although not a primary anti-viral agent, hydroxyurea inhibits HIV-1 replication by indirectly blocking reverse transcriptase, which is dependent on intracellular dNTPs as substrates (Lori, F. et al., Science 266: 801, 1994; Gao, W. Y., et al., Proc. Natl. Acad. Sci. 90: 8925-8928, 1993; Meyerhans, A., et al., J. Virol. 68: 535-540, 1994). Several clinical studies have demonstrated its in vitro and in vivo efficacy for inhibiting HIV-1. Nevertheless, there are studies that suggest that hydroxyurea may also inhibit target cells, such as CD4+ T-lymphocytes, availability as a target, thus accounting for this agent's in vivo anti-retroviral activity (DeBoer, R. J. et al., AIDS 12: 1567-1570, 1998).

Preliminary studies have shown that hydroxyurea-containing regimens profoundly inhibit viral replication if started during primary HIV-1 seroconversion. Of note, at least one patient in a small cohort had a dramatically low proviral reservoir in the peripheral blood, when treated with a ddI and hydroxyurea-containing regimen (Lisziewicz, J. et al., Lancet 352: 199, 1998). In addition, a small cohort of patients on ddI and hydroxyurea did not manifest viral rebound when the anti-viral therapy was discontinued (Vila, J. et al., Lancet 2: 88-89, 1997).

The present invention reveals the effects of hydroxurea on HIV-1 reservoirs in patients on HAART with no detectable viral RNA in the peripheral blood. Of note, hydroxyurea is a relatively small molecule which will cross the blood:brain barrier (Lori, F. et al., Science 266: 801, 1994; Gao, W. Y., et al., Proc. Natl. Acad. Sci. 90: 8925-8928, 1993; Meyerhans, A., et al., J. Virol. 68: 535-540, 1994; DeBoer, R. J. et al., AIDS 12: 1567-1570, 1998) and, therefore, will cross the blood:testes barrier. Additionally, this molecule will also dramatically inhibit the reverse transcription of partial reverse transcripts' processing to full-length reverse transcripts for integration into the host genome (Lori, F. et al., Science 266: 801, 1994). If reverse transcription is normally delayed in certain cells' genital tract reservoirs, as it has been shown to be in many monocytes/macrophage populations due to initially low intracellular dNTP pools (Collin, M., Gordon, S., Virology 200: 114-120, 1993; O'Brien, W. A., et al., J. Virol. 68: 1258-1263, 1994), then treatment with hydroxyurea will further alter this sequential process and decrease proviral integration (based on a lack of fully reverse transcribed viral DNA intermediates). As such, this implies that hydroxyurea will decrease or ablate the development of reservoirs for HIV-1 provirus and replication-competent viruses. This will be especially important if “cryptic” replication takes place in these cells in vivo.

Intensification Therapeutics

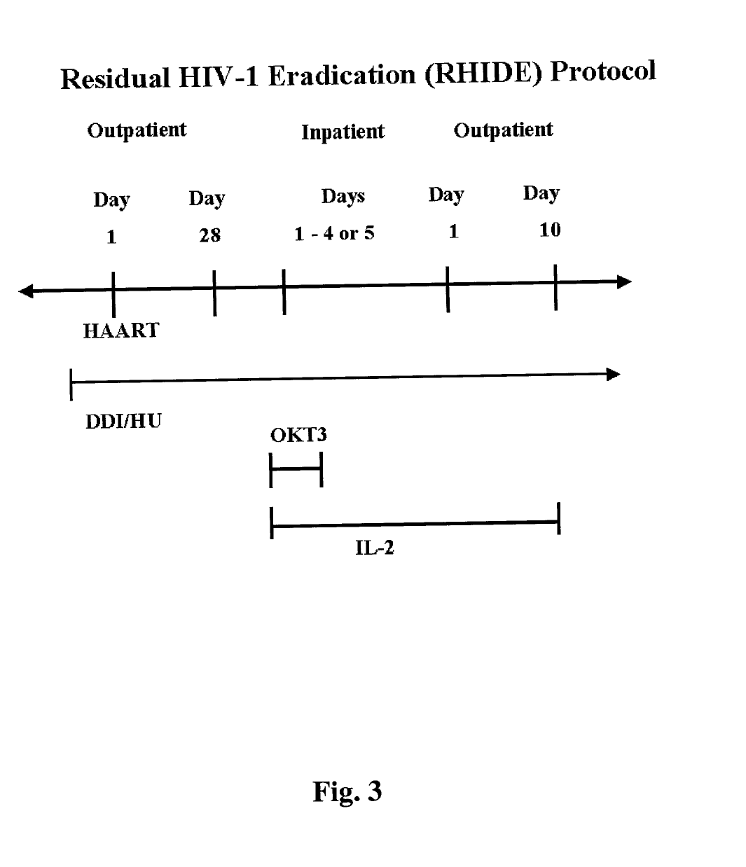

Cytoreduction therapy will also be useful in removing proviral-containing cells. This approach is based on the “prey and predator” relationship of HIV-1 with CD4+ T-lymphocytes. If low levels of ongoing “cryptic replication” occurs in many HIV1-infected-individuals on effective HAART, then “intensification” therapeutics may be added to initial therapy to ablate this low level replication. Compounds that are used as a low level cytotoxic agent in intensification therapeutics include, but are not limited to, hydroxyurea and cyclophosphamide. Further studies on the penetration of different pharmacological agents across blood:tissue barriers (e.g., blood:brain and blood:testes) will be of critical importance in attacking potential sanctuary sites. The present invention includes combinations of each of these approaches (supra) for eradication of HIV-1 in vivo. As such, one might begin to think of the treatment of HIV-1 as somewhat of an “oncological” paradigm. This would include effective HAART as “induction” therapy and then further approaches against HIV-1 latency, cryptic replication and sanctuary sites as analagous to the removal of “residual disease” (FIG. 3).

Hydroxyurea and ddI—a Synergistic Approach to Therapeutics

It has been determined that ddI is the most important reverse transcriptase inhibitor to combine with hydroxyurea (Lori, F. et al., Science 266: 801, 1994; Malley S. D., et al., Proc. Natl. Acad. Sci. 91: 11017-11021, 1994; Gao, W. Y., et al., Molec. Pharm. 46: 767-772, 1994; Malley, S. D., et al., Lancet 343: 1292, 1994). As synergy has been shown with reverse transcriptase inhibitors and hydroxyurea in inhibiting HIV-1, it is critical to add combination therapy when treating with hydroxyurea (Malley S. D., et al., Proc. Natl. Acad. Sci. 91: 11017-11021, 1994; Gao, W. Y., et al., Molec. Pharm. 46: 767-772, 1994). Hydroxyurea decreases dNTP concentrations, this favors incorporation of ddNTPs into proviral DNA. (Lori, F., Science 266:801, 1994). Hydroxyurea decreases dATP concentrations to a greater degree than other dNTPs (Gao, W. Y., et al., J. Clin. Invest. 91: 2326-2333, 1993; Bianchi, V., et al., J. Bio. Chem. 261: 16037-16042, 1986). Thus, the nucleoside reverse transcriptase inhibitor with the greatest potential for synergistic action with hydroxyurea is ddI, which is phosphorylated to ddATP intracellularly. Furthermore, it has been demonstrated that ddI is phosphorylated to ddATP in initially-quiescent CD4+ T-lymphocytes to a greater extent than other nucleoside analogs (Malley, S. D., et al., Lancet 343: 1929, 1994). As such, ddI is critical in synergistic approaches to eradicate HIV-1 infection in initially-resting peripheral blood mononuclear cells (PBMC). The synergy of ddI and hydroxyurea has been clearly demonstrated both in in vitro mechanistic studies, as well by as in vivo analyses (Giacca, M. S., et al., J. Infect. Dis. 174: 204-209, 1996; Rutschmann, O. T., et al., AIDS 12: F71-F77, 1998; Lori, F., et al., Science 266: 801, 1994; Gao, W. Y., et al., Proc. Natl., Acad. Sci. 90: 8925-8928, 1993; Malley, S. D., et al., Proc. Natl. Acad. Sci. 91: 11017-11021, 1994; Gao, W. Y., et al., Molec. Pharn. 45: 767-772, 1994; Malley, S. D., et al., Lancet 343: 1292, 1994; Malley, S. D., et al., Lancet 343: 1929, 1994). The use of abacavir and mycophenolic acid also have profound and synergisitc anti-HIV-1 effects (Margolis, D., et al., Acq Immune Def Syndr 21:362-370, 1999). The use of these compounds is also contemplated as an intensification protocol prior to the stimulation protocol (infra).

Only now are critical questions being addressed regarding the potential eradication of this human retroviral disease. This has been led by both studies of in vivo molecular pathogenesis and the rational design of critical combinations of anti-retrovirals. Nevertheless, small numbers of cells, still remaining in most infected-individuals on effective HAART, are difficult to attack. These cells will clearly “re-ignite” viral replication in many patients who have had HAART withdrawn. These proviral-containing cells in genital secretions will have significant public health importance, as sexual transmission remains possible in all patients with HIV-1 infection. While dramatic strides have been made in the treatment of HIV-1 infection, the present invention provides a novel therapeutic approach to potentially eradicate HIV-1 disease states.

Specifically, the present invention relates to the use of, but not limited to, hydroxyurea and ddI to ablate any low level replication and further to the use of compounds, including, but not limited to, interleukin-2 (IL-2), OKT3 antibodies and γ-interferon, to activate latently-infected cells. The activation of latently infected cells results in the re-initiation of viral replication, these replicating viruses are now susceptible to therapeutic agents. Thus, the invention as disclosed herein provides a novel therapeutic regimen to eliminate all HIV-1 infected cells, with the subsequent eradication of HIV-1.

OKT3

Orthoclone OKT3 (Ortho Biotech, Inc.) is an anti-CD3 monoclonal antibody which has been utilized to reverse rejection of solid tumor allografts, and for T-lymphocyte depletion in bone marrow transplantation (Kreis, H., et al., Transplant Rev. 5: 181-199, 1991; Alam, A., et al., Blood 90: (10) Suppl. 1, 1997). It is indicated for acute allograft rejection in renal transplant patients and for steroid-resistant acute allograft rejection in cardiac and hepatic transplant patients. OKT3 is a purified murine monoclonal antibody directed to the CD3 antigen of human T-lymphocytic cells. This moiety represents an IgG₂ₐ immunoglobulin with a heavy chain of approximately 50,000 daltons and a light chain of approximately 25,000 daltons, with a total molecular weight of 150,000 daltons. The specific binding of OKT3 to the glycoprotein T-cell receptor/CD3 complex (20,000 daltons) interferes with cell-mediated immune responses, possibly blocking the function of T-lymphocytes involved in recognition of foreign antigens (Kreis, H., et al., Transplant Rev. 5: 181-199, 1991). This is the mechanism by which this compound has been used to inhibit acute allograft rejection (Kreis, H., et al., Transplant Rev. 5: 181-199, 1991). Of note, binding of OKT3 to T-lymphocytes results in early activation of these cells, which leads to a cytokine cascade release (Ambramowica, D., et al., Transplantation 47: 606-608, 1989; Chatenoud, L., et al., Transplantation 49: 69-702, 1990; Chatenoud, L., et al., Transplantation 51: 334-338, 1991; Chatenoud, L., et al., N. Engl. J. Med. 320: 1420-1421, 1989; Ferran C., et al., Eur. J. Immunol. 20: 509-515, 1990; Ellenhorn, J. D. I., et al., J. Immunol. 144: 2840-2846, 1990; Ellenhorn, J. D. I., et al., Transplantation 50: 608-612, 1990; van Seventer, G. A., et al., J. Immunol. 139: 2545-2550, 1987; Hirsch, R., et al., J. Immunol. 142: 7373-7343, 1989; Hirsch R., et al., J. Immunol. 140: 3766-3772, 1988; Spits, H., et al., Eur. J. Immunol. 15: 88-92, 1985), and then transient T-lymphocyte depletion. T-lymphocyte function may be altered by this treatment, but within one week of terrnination of OKT3 in solid organ transplant subjects, T-cell function returns to baseline levels.

Elevated levels of tumor necrosis factor alpha (TNF-α, gamma interferon (IFN-γ), and interleukin-2 (IL-2) are demonstrated in the initial treatment of patients with OKT3 (Chatenoud, L., et al., Transplantation 49: 69-702, 1990). Over 24 hours, the elevation in these critical cytokines diminishes to normal levels. This is, nevertheless, accompanied by a series of side-effects that may include fever, chills, headache, vomiting, diarrhea and potentially hypotension and bronchospasm (Kreis, H., et al., Transplant Rev. 5: 181-199, 1991). These clinical manifestations usually decrease after the first treatment, concomitant with the decrease in cytokine release after the first treatment. Corticosteroids have been shown to inhibit, to some degree, the release of these cytokines during treatment with OKT3 (Chatenoud, L., et al., Transplantation 49: 69-702, 1990,Chatenoud, L., et al., Transplantation 51: 334-338, 1991).

In addition to treatment of allograft rejection and T-lymphocyte depletion during bone marrow transplantation, OKT3 has also begun to be evaluated for its ability to stimulate T-lymphocytes and generate a cytokine cascade in several disease states. Studies have been reported using OKT3 to stimulate CD4+ T-lymphocytes in vitro in patients treated for advanced cancer (Curti, B. D., et al., J. Clin. Oncol. 16: 2752-2760, 1998). These in vitro stimulated CD4+ T-lymphocytes were then re-infused into patients who were also treated with intravenous (IV) IL-2, and cyclophosphamide (Curti, B. D., et al., J. Clin. Oncol. 16: 2752-2760, 1998).

OKT3 activates T-lymphocytes

Some studies have now begun to evaluate low doses of OKT3 in attempts to stimulate T-lymphocytes, without leading to T-lymphocytic depletion. In a recent study (Urba, W. J., et al., Cancer Res. 52: 2394-2401, 1992), OKT3 was utilized at various microgram levels, rather than the 5 mg treatment doses per day (usually 5 day courses) utilized in allograft rejection therapy. High doses of OKT3 have been noted to lead to both CD3-induced immunostimulatory effects and T-lymphocyte activation with proliferation properties, as well as cytokine production and the induction of antigen-specific and non-specific cytotoxic T-lymphocytes. In contrast, Urba, et al., demonstrated that treatment of patients with 1 to 100 microgram infusions results in lower levels of cytokine-induced clinical side-effects.

In addition to cytokine production (supra), recent studies have also shown that OKT3 may stimulate granulocyte-macrophage colony stimulating factor (GM-CSF), interleukin-6 (IL-6), and lead to increased interleukin-2 receptor (IL-2R) levels, and increased mixed lymphocyte reactions (Abramowica, A., et al., Transplantation 47: 606-608, 1989; Urba, W. J., et al., Cancer Res. 52: 2394-2401, 1992). The study by Urba, et al. (supra) suggests that at lower doses anti-CD3 can result in apparent stimulation of T-lymphocytes in vivo. Lower doses were also found to have less of the undesirable side-effects and profound immunosuppression found after multiple doses of high level OKT3. Of note, though, headache and aseptic meningitis were shown in this study to be more of an important side-effect, as compared to previous studies using higher doses (Urba, W. J., et al., Cancer Res. 52: 2394-2401, 1992). Analysis of patients treated in this study suggested that many of the microgram doses of OKT3 led to actually suboptimal in vivo T-lymphocyte activation. Nevertheless, 100 micrograms of OKT3 seemed to demonstrate some significant T-cell activation parameters in vivo. The authors, though, conclude that using OKT3 at these lower levels actually led to excessive toxicity, especially the severe headaches and aseptic meningitis, without dramatic increases in T-cell activation, as previously described by others (Abramowica, A., et al., Transplantation 47: 606-608, 1989), when utilized in vivo in patients with tumors (Urba, W. J., et al., Cancer Res. 52: 2394-2401, 1992).

In recent data from J. Bluestone et al., low doses of humanized anti-T3 antibody (200-400 μg) were demonstrated to activate T-lymphocytes, without leading to T-lymphocyte depletion. Of note, only one treatment was possible, as T-lymphocyte depletion occurs, even at this low dose, if more than one injection (i.e., daily injections for 5 days) is administered.

In studies by Sosman, et al., (Sosman, J. A., et al., J. Clin. Oncol. 11: 1496-1505, 1993; Sosman, J. A., et al., J. Immunotherapy 17: 171-180, 1994), OKT3 at low doses (10-600 μg per m² over 5 to 15 minutes on the first day of therapy) was followed with either high-dose bolus IL-2 (89) or low dose continuous infusion of IL-2 (Sosman, J. A., et al., J. Immunotherapy 17: 171-180, 1994), in patients with various malignancies. No significant evidence for T-lymphocyte activation which was OKT3 dose-dependent, save for increased soluble IL-2 receptor levels, was found in one study (Sosman, J. A., et al., J. Clin. Oncol. 11: 1496-1505, 1993). In this study (Sosman, J. A., et al., J. Clin. Oncol. 11: 1496-1505, 1993), transient fever, chills, confusion and hypotension were noted but no headache or neck stiffness. The maximum tolerated dose (MTD) was 400 μg/m². The lower doses of OKT3 used in this study were based on animal studies, which suggested that higher doses led to T-lymphocyte depletion (Ellenhom, J. D. I., et al., Science 242: 569-571, 1989; Hirsch R., et al., J. Immunol. 142: 737-742, 1989; Hirsch R., et al., J. ImmunoL 140: 3766-3772, 1988). Of note, OKT3 was administered one day prior to IL-2, to stimulate T-cell activation and IL-2 receptor expression, which is not induced by IL-2 alone. (Lotze, M. T., et al., Cancer Res. 47: 2188-2195, 1987).

In the second study by Sosman (Sosman, J. A., et al., J. Immunotherapy 17: 171-180, 1994), clear signs of T-cell activation were again not statistically significant. This agreed with a small trial of low dose OKT3 and subcutaneous IL-2 in patients with metastatic tumor, by Buter, et al., (Butler, J., et al., Eur. J. Cancer 29: 2108-2113, 1993). This lack of immune activation may be secondary to T-cell receptor signaling dysfunction in many cancer patients (Weil-Hillman, G. K., et al., J. Immunother. 10: 267-277, 1991; Mizoguchi, H., et al., Science 258: 1795-1798, 1992; Finke, J. H., et al., Cancer Res. 53: 5613-5616, 1993; Nakagomi, H., Cancer Res. 53: 5610-5612, 1993). A re-distribution of activated T-lymphocytes to lymphoid tissues remains quite possible.

A very recent study from J. Bluestone (Richards, J., et al., Cancer Res. [In Press] ) utilizing a “humanized” OKT3 antibody in cancer patients, revealed its potential utility at low doses (50-1600 μg/patient) to activate T-lymphocytes in vivo. Side-effects including headache, fever and rigors were moderate and transient up to a dose of 800 μg. Several patients demonstrated increased soluble IL-2 receptor and IL-6 levels after treatment with doses of 400 μg and above. As well, at the 400 μg dose, the T-lymphocyte counts were 75% of baseline at 24 hours and returned to baseline at 48 hours. (Richards, J., et al., Cancer Res. [In Press]).

OKT3 Induces HIV-1 Viremia

It has been demonstrated in a short letter that OKT3 can induce HIV-1 viremia, probably by stimulating T-lymphocytes, in a patient infected with HIV-1 (Brinkman, K., et al., Lancet 352: 1446, 1998). As well, based on these data, Lange's group has recently treated three HIV-1-positive patients on HAART with combined IL-2 and OKT3 (Prins, J., et al., 6^{th} Conference on Retroviruses and Opportunistic Infections, Chicago, Ill. February 1999, LB6; VanPraag, M., et al., 6^{th} Conference on Retroviruses and Opportunistic Infections, Chicago, Ill. Feb. 1999, #29). These patients demonstrated high levels of cytokine release and T-lymphocyte activation. HIV-1 levels in the peripheral blood plasma increased to detectable levels, even though HAART was continued during this trial. In these three patients, no clear change in the ability to isolate replication-competent virus from peripheral blood CD4+ T-lymphocytes was noted. The authors also note important toxicity of OKT3 combined with IL-2 (2-4.5×106 IU sq qday) in these patients based on the cytokine release syndrome, which has been characterized previously (Abramowica, D., Transplantation 47: 606-608, 1989). Of note, the increased viral replication became undetectable after the OKT3/IL-2 treatment was discontinued. It is important to note that in this trial OKT3 was utilized at 5 milligrams IV qday for 5 days, and no intensification therapy had been added previously to the patients' HAART regimens (Prins, J., et al., 6^{th} Conference on Retroviruses and Opportunistic Infections, Chicago, Ill. February 1999, LB6; VanPraag, M., et al., 6^{th} Conference on Retroviruses and Opportunistic Infections, Chicago, Ill. February 1999, #29).

The present invention provides a therapeutic regimen which includes OKT3 for stimulation of CD4+ T-lymphocytes in HIV-1-infected patients on HAART with undetectable virus in their peripheral blood. Due to the cytokine release syndrome secondary to infusion of OKT3, other latent reservoirs, including macrophages may be stimulated by the released interferon gamma and GM-CSF. Of importance, human anti-mouse antibodies (HAMA) may be a potential problem if more than a one course of OKT3 is to be used in therapy of HIV-1-infected-individuals (Kreis, H., et al., Transplant Rev. 5: 181-199, 1991).

Interleukin-2 (IL-2)

Interleukin-2 (IL-2) is a cytokine which possesses significant immuno-modulating effects. These include stimulation and proliferation of CD4+and CD8+T-lymphocytes, increased cytotoxtic T-lymphocyte enhancement, stimulation of monocyte/macrophages via induction of interferon gamma and increased antibody production (Jacobson E. L., Proc. Natl. Acad Sci. 93: 10404-10410, 1996). As such, this cytokine has generated significant interest for potential therapy of HIV1-infected-individuals. IL-2 has a molecular weight of 15,300 daltons and is produced via recombinant DNA technology using genetically-engineered E. coli (Proleukin; Chiron, Corp.) An initial series of studies suggested that intravenous or subcutaneous IL-2, at both high (18 million IU per day) and low doses (60 to 250,000 IU per day), can lead to immunostimulatory effects in patients infected with HIV-1 and manifesting CD4+ T-lymphocyte depletion (Schnittman, S. M. et al., J. Infect. Dis. 169: 981-989, 1994; Ramachandran, R., et al., J. Infect. Dis. 173: 5-8, 1996; Teppler, H., et al., J. Infect. Dis. 167: 291-298, 1993; Teppler, H., et. al., J. Exp. Med. 177: 483-492, 1993; Wood, R., J. Infect. Dis. 167: 519-525, 1993).

IL-2 in Low Doses

In initial studies by Teppler, et al., (Teppler, H., et al., J. Infect. Dis. 167: 291-298, 1993; Teppler, H., et. al., J Exp. Med. 177: 483-492, 1993), low doses of polyethylene glycol-conjugated (PEG) IL-2 administered subcutaneously at low doses led to increased delayed type hypersensitivity (DTH) responses in initially anergic patients with HIV-1 infection. Effects on CD4+ T-lymphocyte percentages and lymphocyte blast transformation were also demonstrated in a study by Schnittman, et al., (Schnittman, S. M., et al., J. Infect. Dis. 169: 981-989, 1994), combining IL-2 with interferon-alpha 2b. When in combination with thymosin-alpha, IL-2 led to similar immunomodulatory effects (Ramachandran, R., et al., J. Infect. Dis. 173: 5-8, 1996). Further studies by Jacobson, et al., (Jacobson, E. L., et al., Proc. Natl. Acad. Sci. 93: 10405-10410, 1996) confirmed the utility of low dose IL-2 (60 to 250,000 IU per m² per day given subcutaneously) in inducing immunostimulation and increased DTH responses in HIV-1-infected-individuals.

Most recently, “ultra low” doses of IL-2 (1.2×10⁶ IU/m²/day) demonstrated significant immunostimulatory effects and normalization of interferon gamma production in HIV-1-infected-individuals (Khatri, V. P., et al., J. Clin. Invest. 101: 1373-1378, 1998).

High Doses of IL-2

Studies by Davey, et al., have shown that subcutaneous administration of IL-2 at higher doses (3 to 18 million IU per day) led to increases in CD4+ T-lymphocytes in patients with HIV-1 infection and CD4+ T-lymphocyte counts greater than 200/mm³ (Davey, R. T., et al., J. Infect. Dis. 175: 781-789, 1997). In this study, it was noted that plasma viral load increased transiently after each injection of IL-2.

A second group of recent studies have utilized IL-2 in attempts to dramatically increase CD4+ T-lymphocyte counts and function in patients infected with HIV-1 (Kovacs, J. A., et al., N. Engl. J. Med. 332: 567-575, 1995; Kovacs J. A., et al., N. Engl. J. Med. 335: 1350-1356, 1996). Relatively high doses of IL-2 given intravenously at 18×10⁶ IU/per day (every two months, five consecutive day treatments for 6 cycles) led to profound increases in CD4+ T-lymphocytes in HIV-1-infected-individuals with baseline CD4+ T-lymphocyte counts greater than 200 cells/mm³ (increases of approximately 400 cells per mm3).

Hengge, et al., (Hengge, U. R., et al., AIDS 12: F225-F234, 1998) showed that five day cycles of subcutaneous IL-2 (9×10⁶ IU per day) led to significant increases in CD4+ T-lymphocyte counts in patients on HAART, as compared to patients treated with HAART alone. Carr, et al., (Carr, A., et al., J. Infect. Dis. 178: 992-999, 1998) demonstrated more significant increases in CD4+ T-lymphocytes in HIV-1-infected-individuals treated with IV IL-2 (12×10₆ IU per day), as compared to PEG-IL-2 given subcutaneously (6.5×10⁶ IU per day).

These studies (supra), therefore, show that IL-2 can generate both increased immunostimulatory effects, as well as an increase in CD4+ T-lymphocyte levels, when used in the treatment of HIV-1-infected-individuals. This is interesting in light of recent data demonstrating an inhibition of T-lymphocyte production by HIV-1 in vivo (Hellerstein, M., et al., Nature Med 5: 83-89, 1999).

IL-2 Decreases Replication-competent Virus from Resevoirs

Recent reports from the Fauci and Lane groups suggest that use of IL-2 may also decrease the levels of replication-competent virus from proviral reservoirs in patients infected with HIV-1 on effective HAART (Chun, T. W., et al., et al., Nature Med 5:651-655, 1999; Imarnichi, H., et al., 6^{th} Conference on Retroviruses and Opportunistic Infections, Chicago, Ill., February 1999, #358). As such, IL-2 has now potentially three critical effects for treatment of HIV-1-infected-individuals. These include, but are not limited to: 1) increased immunomodulating effects, 2) increased CD4+ T-lymphocyte counts, and 3) depletion of proviral reservoirs (Schnittman, S. M., et al., J. Infect. Dis. 169: 981-989, 1994; Ramachandran, R., et al., J. Infect. Dis., 173: 5-8, 1996; Teppler, H., et al., J. Infect. Dis. 167: 291-298, 1993; Teppler, H., et al., J. Exp. Med. 177: 483-492, 1993; Wood R., et al., J. Infect. Dis. 167: 519-525, 1993; Davey, R. T., et al., J. Infect. Dis. 175: 781-789, 1998; Kovacs , J. A., et al., N. Engl. J. Med. 332: 567-575, 1995; Kovacs, J. A., et al., N. Engl. J. Med. 335: 1350-1356, 1999; Hellerstein, M., et al., Nature Med. 5: 83-89, 1999; Chun, T. W., et al., et al., Nature Med 5:651-655, 1999; Imamichi, H., et al., 6^{th} Conference on Retroviruses and Opportunistic Infections, Chicago, Ill., February 1999, #358; Hengge, U. R., et al., AIDS 12: F225-F234, 1998; Carr, A., et al., J. Infect. Dis. 178: 992-999, 1998). While decreases in replication-competent virus were initially detected, all patients had a rebound after stopping HAART, even while on the IL-2 treatment alone regimen. (Davey, RT., et al., PNAS 96:15109-10114, 1999). Further, none of these studies used HAART with new additions of intensification therapeutics during IL-2 treatment, or in treatment with OKT3 and IL-2 combined, as in the present invention. Therefore, if IL-2 is utilized in patients on HAART, and the transient increases in viral RNA are not inhibited, these viruses may infect previously uninfected T-lymphocytes (“predator vs prey relationship”). This would “reset the virological clock” and result in new on-going infections of CD4+ T-lymphocytes. Of importance, these trials by Kovacs, et al., (Kovacs, J. A., et al., N. Engl. J. Med. 332: 567-575, 1995; Kovacs, J. A., et al., N. Engl. J. Med. 335: 1350-1356, 1996) did not fully evaluate patients on HAART and solely noted that increased viral replication was especially common in patients with less than 200 CD4+ T-lymphocytes per mm3.

IL-2 Treatment of Patients on HAART

At a recent conference on retroviruses and opportunistic infections, several other studies using IL-2 in patients with HIV-1 infection were reported in (Losso, W., et al., 6^{th} Conference on Retroviruses and Opportunistic Infections, Chicago, Ill., February 1999, #354; Tambussi L., et al., 6^{th} Conference on Retroviruses and Opportunistic Infections, Chicago, Ill., February 1999, #355; Stellbrink, H. J., et al., 6^{th} Conference on Retroviruses and Opportunistic Infections, Chicago, Illinois, February 1999, #356; Davey R., et al., 6^{th} Conference on Retroviruses and Opportunistic Infections, Chicago, Ill., February 1999, #357; Siegla, J. P., Puri, R., K. J. Clin. Oncol. 9: 694, 1991). In a modest size trial of 37 patients, approximately 50% on HAART, infected-individuals were treated with IL-2 (1.5 to 7.5 million IU sq per day), which led to significant increases in the CD4+ T-lymphocytes without significant changes in plasma RNA (Losso, W., et al., 6^{th} Conference on Retroviruses and Opportunistic Infections, Chicago, Ill., February 1999, #354). A small trial of low versus high dose IL-2 demonstrated increases in CD4+ T-lymphocytes in patients on HAART. No significant increases in viremia were noted and subcutaneous IL-2 at low doses (3×10⁶ IU bid, 5 days every 4 weeks) was shown to be the best tolerated regimen (Tambussi, L., et al., 6^{th} Conference on Retroviruses and Opportunistic Infections, Chicago, Ill., February 1999, #355). In a small trial of patients on HAART, IL-2 therapy (9×10⁶ IU day subcutaneously, 5 days every 6 weeks) demonstrated increases in total CD4+ T-lymphocytes and CD4+ CD45RA positive cells in vivo (Stellbrink, H. J., et al., 6^{th} Conference on Retroviruses and Opportunistic Infections, Chicago, Ill., February 1999, #356). No significant increase in plasma HIV-1 RNA was demonstrated.

Subcutaneous IL-2 Administration

An abstract by Davey, et al. showed in a multi-center trial that subcutaneous IL-2 (5 day courses every 8 weeks for 6 cycles at a starting dose of 7.5×10⁶ IU bid) led to significant increases in CD4+ T-lymphocyte counts (mean values of 355 at entry, 739 after treatment) (Davey, R., et al., 6^{th} Conference on Retroviruses and Opportunistic Infections, Chicago, Ill., February 1999, #357). Of importance, subcutaneous IL-2 treatment has demonstrated significant increases in CD4+ T-lymphocyte levels within HIV1-infected-individuals with less severe toxicities and fewer capillary leak syndromes, as compared with treatment using intravenous IL-2 (Jacobson, E. L., et al., Proc. Natl. Acad. Sci. 93: 10405-10410, 1996; Davey, R. eet al., J. Infect. Dis. 175: 781-789, 1997; Hengge, U. R., et al., AIDS 12: F225-234, 1998).

H.C. Lane's group also demonstrated that intermittent five day administrations of IL-2 again led to increases in CD4+ T-lymphocyte counts with HIV-1 infection (Imamichi, H., et al., 6^{th} Conference on Retroviruses and Opportunistic Infections, Chicago, Ill., February 1999, 358). In several of these patients, a transient rise of plasma viral RNA was noted. In a preliminary study by this group, the HIV-1 quasispecies expressed in the blood plasma during IL-2 therapy did not appear to match with PBMC proviral DNA sequences (Imamichi, H., et al., 6^{th} Conference on Retroviruses and Opportunistic Infections, Chicago, Ill., February 1999, 358). This level of augmentation of viral replication was noted to be transient in the studies of patients whom were not on effective HAART therapy. Finally, a very recent report has demonstrated that IL-2 decreases the HIV-1 proviral load in PBMCs and increases CD4+, CD45 RA+T-lymphocytes in certain infected-individuals (Zanussi, S., et al., AIDS Res and Human Retroviruses 15: 97-103, 1999).

A Novel Therapeutic Approach to Eradication HIV-1

Human immunodeficiency virus type I (HIV-1) infections have been remarkably altered by the advent of highly active anti-retroviral therapy (HAART), in which combination chemotherapy is utilized. These therapies have allowed significant proportions of HIV-1-infected populations in the developed world to obtain and maintain undetectable viral loads in their peripheral blood plasma. These therapies have led to significant effects on mortality and morbidity. Nevertheless, persistently-infected cells containing proviral DNA, and able to reproduce replication-competent virus in vitro, are found in the vast majority of these patients. As such, V-1 has not been eradicated in patients on HAART with undetectable load in their blood plasma.

The present invention relates to a novel approach to HIV-1 therapeutics (FIG. 3). An “intensification” therapy with hydroxyurea/ddI will inhibit the development of any HIV-1 resevoirs from developing. By inhibiting the completion of viral transcription of any partially transcribed viruses (“cryptic” replication), the amount of viruses able to integrate into the host cells genome decreases. This regimen is followed by a “stimulation” regimen wherein the stimulation of latently-infected cells with OKT3 and IL-2 occurs. This results in a “re-infection”/stimulation of the latently-infected cells by the virus. The replication of the latent virus that results from this “re-infection” is then inhibited with the hydroxyurea/ddI therapeutics, thereby leading to eradication of HIV-1 in vivo in selected patients. This amounts to a two-pronged approach to both inhibit “cryptic” replication and stimulate latently-infected T-lymphocytes +/− monocyte/macrophages. The intensification therapy is critical during the “stimulation phase” to inhibit HIV1 replication. A combined stimulation protocol of OKT3 with IL-2 replicates the approach utilized to stimulate HIV-1 growth in vitro (O'Brien W., Pomerantz, R. J. Viral Pathogenesis. New York: Raven Press, 813-837,1997).

The present invention combines intensification therapy using hydroxyurea and ddI to inhibit cryptic replication that in PBMC's, lymphoid cells and sanctuary sites, such as the male genital tract and central nervous system. The hydroxyurea/ddI therapeutic regimen has critical effects on resting cells and therefore HIV-1 persistence mechanisms (supra). The present invention further stimulates latently-infected cells, thereby initiating the re-infection. These “newly” infected cells are now suseptible to inhibition by HAART and the patients' immune system. The therapeutic regimen of the present invention is therefore a novel approach towards complete eradication of HIV-1 infections in vivo in selected patients.

While this invention is described with a reference to specific embodiments, it is obvious to those of ordinary skill in the art that variations in these compounds and methods may be used and that it is intended that the invention may be practiced otherwise than as specifically described herein. Accordingly, this invention includes all modifications encompassed within the spirit and scope of the invention as defined by the claims.

Methods

Coculture Assay

CD8+ T-lymphocytes are depleted from the isolated PBMC by binding to magnetic beads conjugated with anti-CD8 antibody (Biosource, Camarillo, Calif). This process decreases the fraction of CD8+ T-lymphocytes in the PBMC from approximately 20%-30% to 3%-5%, as analyzed by flow cytometry. Depletion of CD8+ T-lymphocytes significantly increases in vitro outgrowth of HIV-1 from the PBMC (Zack, J. A., et al., Cell 61: 213-225, 1990; Zack, J. A., et al., J. Virol. 66: 1717-1725, 1992; Bukrinsky, M. I., et al., Science 254: 423-427, 1991) because CD8+ cells secrete chemokines and other factors that inhibit the replication of the virus (Wei, X., et al., Nature 3: 1177-122, 1995) Macrophages and their precursors are depleted from PBMC by incubating the samples overnight to allow these cells to attach to the plastic plates. The remaining PBL are then stimulated with 5 μg of phytohemagglutinin per milliliter (Sigma, St. Louis) and 50 U of interleukin-2 (IL-2) per milliliter (GIBCO-BRL, Grand Island, N.Y.). PBL are isolated from blood samples obtained from normal subjects with the same procedure. The PBL from the patients are then mixed in a 1:1 ratio with those from normal subjects (2 million cells each) and cultured in RPMI-1640 medium with 10% fetal calf serum and penicillin plus streptomycin at 37° C. for six weeks. Twice a week, half the medium is replaced with fresh medium. Once a week, half the cells are replaced with 2 million fresh PBL from normal subjects after stimulation with phytohemagglutinin and IL-2 and depletion of CD8+ T-lymphyocytes.

The serninal-cell pellet is washed twice with cold phosphate buffered saline (PBS), and 3 million cells are mixed with 2 million PBL from normal subjects after the depletion of CD8 T lymphocytes and stimulation with phytohemagglutinin and IL-2. After 24 hours, the cells are washed three times with PBS, and the cultures are maintained in the presence of IL-2 (10 U per milliliter) for six weeks. Twice a week, half of the medium is replaced with fresh medium. Once a week, the cells are replenished with 2 million fresh, treated PBL from normal subjects. HIV-1 p24 antigen is measured in the supernatants by an enzyme-linked immunosorbent assay (ELISA) (Dupont, Wilmington, Del.). All procedures are performed under level P3 biosafety conditions to minimize the possibility of cross-contamination.

HIV-1 Virion RNA Isolation and Quantitative RT-PCR

The blood plasma and genital fluids are concentrated via ultracentrifugation at 35000 rpm for 1 hour. The surpematant is discarded and virion-associated genomic RNA is extracted from the subsequent pellet using a guanidinium thiocyanate method (Promega Inc, Madison, Wis.) (Stevenson M., et al., EMBO J. 9: 1551-1560, 1990). The isolated RNA is treated with 10 U of RQ1Dnase. The RNA is precipitated with transfer RNA (tRNA, Gibco Inc, Rockville, Md) as a carrier and resuspended in RNase-free water. Using this approach, reverse transcriptase-negative controls led to no detectable bands after PCR, which demonstrated the lack of viral DNA contamination (Pomerantz, R. J., et al., Cell 61: 1271-1276, 1990; Butera S. T., et al., J. Virol. 68: 2726-2730, 1994; Michael, N. P., et al., 65: 1291-1303, 1991).

Twelve microliters of each viral RNA sample (1 mL of plasma or genital fluid equivalent) is mixed with 1 μL of SK39 primer (SEQ. ID. NO: 1) (50 μmol/L) followed by incubation at 55° C. for 20 minutes. Then, 12 μL of a reverse transcriptase cocktail containing 5 μL of 5× reverse transcriptase buffer, 2.5 μL of 100-mmol/L dithiothreitol, 3.5 μL of 3-mmol/L deoxyribonucleoside triphosphates, and 1 μL of Moloney murine leukemia virus reverse transcriptase enzyme (Gibco Inc) is mixed with the RNA sample containing the primer. This mixture is then incubated at 37° C. for 30 minutes. The reverse transcriptase is inactivated by boiling the samples for 10 minutes. The resultant complementary DNA is then analyzed by quantitative PCR with primers SK38 (SEQ. ID. NO: 2) andSK39 (SEQ. ID. NO: 1) to the gag region of the HIV-1 genome for 30 cycles, as described by Zhang et al., (Pomerantz, R. J., et al., Cell 61: 1271-1276, 1990). The amplified PCR products are hybridized with a probe labeled with phosphorus 32, SK19 (SEQ. ID. NO: 3), and Southern blotting is then used to visualize the specific bands of the amplicons (Pomerantz, R. J., et al., Cell 61: 1271-1276, 1990; Butera S.T., et al., J. Virol. 68: 2726-2730, 1994; Michael, N. P., et al., 65: 1291-1303, 1991) A standard curve is developed using an in vitro transcribed gag RNA construct, as described in Zhang et al., (Pomerantz, R. J., et al., Cell 61: 1271-1276, 1990). Comparison of the test samples with this serially diluted standard is used to quantify viral unspliced RNA to 5 copies/mL, within the linear amplification range of this assay. Viral transcripts below 5 copies/mL also are detected using this assay system. Quantitation of the viral transcripts is performed via analysis using a Phospholmager (Molecular Dynamics, Sunnyvale, Calif.).

Human immunodeficiency virus type I (HIV-1) infections have been remarkably altered by the advent of combination chemotherapy (HAART) (supra). Nevertheless, persistently-infected cells containing proviral DNA that are able to produce replication-competent virus in vitro are found in the vast majority of these patients. As such, HIV-1 has not been eradicated in patients on HAART with an undetectable viral load in their blood. In addition to PBNCs and lymphoid cells, seminal cells in infected-men on HAART have recently been shown to also contain proviral DNA and replication-competent viruses. As such, the invention provides an “intensification” therapy using hydroxyurea and didanosine (ddI), in attempts to ablate any “cryptic” replication that may be taking place in blood, lymphoid tissue and other sanctuary sites, including the male genital tract.

The initial therapeutic regimen of the present invention analyzes the efficacy of the initial regimen in twenty-four male patients, all of whom have had sustained undetectable viral RNA in their peripheral blood plasma after initiation of HAART. Twelve will be continued on these regimens, while a randomized set of twelve men will then be started on hydroxyurea and ddI, in addition to their standard HAART. Patients are followed for presence and quantitation of proviral DNA, as well as replication-competent viral isolation, in peripheral blood cells and seminal cells, and residual HIV-1 RNA in plasma (below 50 copies/ml).

While the invention herein is described with a reference to specific embodiments, it will be obvious to those of ordinary skill in the art that variations to these methods may be practiced otherwise that as specifically described herein. Accordingly, the invention includes all modifications encompassed within the spirit and scope of the invention as defined by the claims. Thus, the following are examples of the therapeutic regimen and is in no way meant to limit the invention.

### EXAMPLE 1

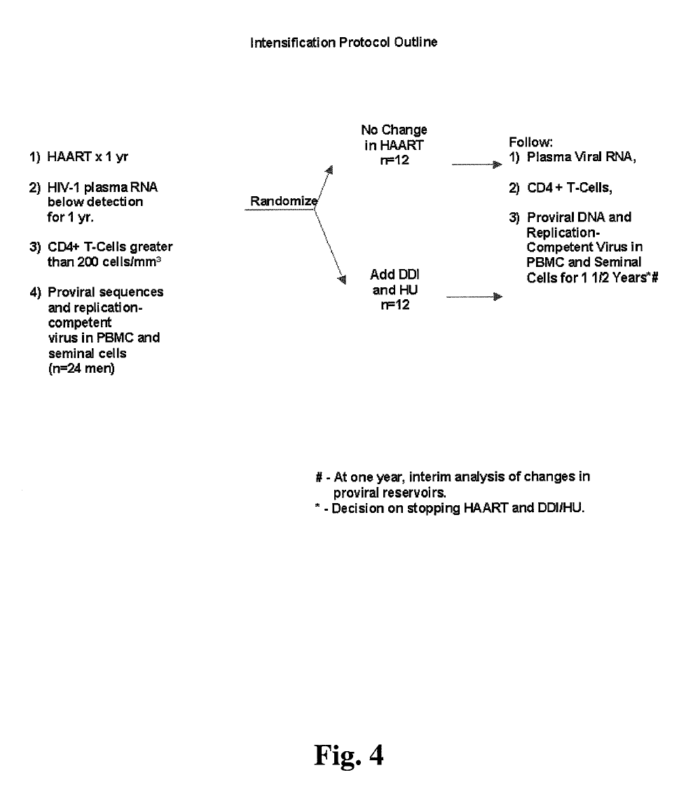

A 78-week study in which 24HIV-1-infected-men on HAART (FIG. 4), but not on ddI or hydroxyurea at the start of this study, with no detectable virus in the peripheral blood for a period of at least one year, is randomized into two groups of twelve. In one group, the standard HAART therapy is continued. In the second group, hydroxyurea and ddI is added to the regimen (i.e., intensification). Hydroxyurea is started at 500 mg bid po (500 mg capsules of Hydria) with ddI 400 mg po qday (as 2 tablets of 200 mg) (250 mg po qday for patients less than 60 kg). The subjects' proviral DNA levels in peripheral blood and seminal samples are monitored on a bimonthly basis. The presence and levels of replication-competent viruses, which is obtained from these samples using co-culture assays with PBMCs from HIV1-seronegative individuals, after CD8+ T-lymphocyte depletion (Zhang, H., et al., The New England Journal of Medicine 339(25):1803-1809, 1998; Dornadula, G., et al., Jama 282(17): 1627-1632, 1999), is analyzed. In addition, a laboratory-based RT-PCR technique with a quantitation limit of 5 copies/ml for HV-1 RNA is used to analyze low copy numbers of residual virion RNA in blood plasma.

Each subject has at least two viral RNA levels below 400-500 copies/ml in the peripheral blood plasma for at least a one year period prior to initiating the therapeutic regimen of the invention, as assessed by reverse transcriptase (RT)-polymerase chain reaction (PCR) (Roche, Inc), or by branched chain DNA analysis (Chiron, Inc., performed as per manufacturers protocol). During the initial screening, each subject has one ultrasensitive test for HIV-1 RT-PCR (supra) (Roche, Inc.) in the undetectable range (below 50 copies/ml) in blood plasma at enrollment. During this study, all subjects' HIV-1 RNA levels are screened using this ultrasensitive methodology to detect any rise in plasma viral RNA above 50 copies per milliliter. Peripheral blood CD4+ T-lymphocyte counts are obtained at the time of enrollment. All men in the study have CD4+ T-lymphocyte counts greater than 200 cells/mm³(infra).

Primary Procedure

1) The safety and tolerability of hydroxyurea and ddI as intensification therapy for patients on HAART with undetectable plasma HIV-1 RNA is determined.

2) The presence and levels of proviral DNA, replication-competent HIV-1 in PBMCs, seminal cells, and free-virion RNA in blood plasma of these patients is analyzed throughout the hydroxyurea and ddI intensification therapy.

Secondary Procedure

1) RT-PCR (limit 5 copies/ml) is used to analyze residual plasma viral RNA. Decreases in proviral sequences and replication-competent viruses from seminal and PBMC cells and/or residual plasma HIV-1 RNA are monitored. The time at which residual viral RNA becomes undetectable on hydroxyurea and ddI, a decision is made as to whether or not to discontinue HAART. If HAART is discontinued, the patient will be monitored clinically and virologically for the development of any disease symptoms (“viral rebound”).

2) The in vivo half-life of proviral containing cells and replication-competent virus levels in PBMCs and seminal cells in patients on ddI and hydroxyurea with HAART, as compared to standard HAART alone, is determined.

### EXAMPLE II

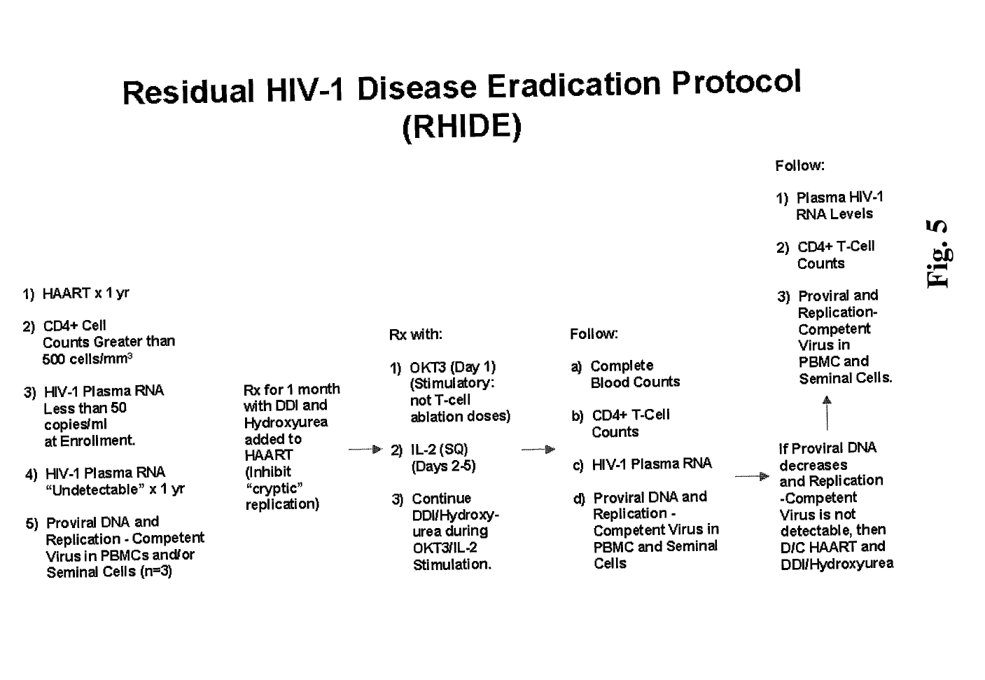

This is a 48 week study in which three HIV-1-infected-men on HAART (FIG. 5), but not on ddI or hydroxyurea at the start of this study, with no detectable virus in the peripheral blood for at least one year, will be treated with novel “intensification” therapy using ddI and hydroxyurea and “stimulation therapy” utilizing OKT3 (Orthoclone OKT3-muroMonab-CD3; Ortho-Biotech, Inc.) and IL-2 (Proleukin; Chiron, Inc.). Each of these patients will be on stable HAART without changes in their antiretroviral regimen for at least one year and will have CD4+ T-lymphocyte counts greater than 500 cells/mm³.

Hydroxyurea and ddI are added to the HAART regimen (i.e., intensification) for one month prior to treatment (supra) with OKT3 and IL-2. Hydroxyurea is started at 500 mg bid po (500 mg capsules of Hydria) with ddI 400 mg po qday (as 2 tablets of 200 mg) (250 mg po qday for patients less than 60 kg). The patients are monitored on this intensification therapy for one month prior to treatment with OKT3 and IL-2. Of note, proviral DNA levels and replication-competent virus are obtained, prior to treatment with ddI/hydroxyurea, via evaluation of PBMCs and seminal cells of these patients. After one month of therapy on ddI and hydroxyurea and prior to treatment with IL-2 and OKT3, the blood and seminal samples are again collected for proviral load and replication-competent virus from seminal cells and PBMCs. The presence and levels of replication-competent viruses which are obtained from these samples, using co-culture assays with PBMCs from HIV-1-seronegative individuals after CD8+ T-lymphocyte depletion, are determined (Zhang, H., et al., N. Engl. J. Med. 339(25): 1803-1809, 1998; Domadula, G., et al., JAMA 282(17): 1627-1632, 1999).

The course of OKT3 and IL-2 will mimic in vitro activation of PBMCs, for growth and expression of HIV1. As IL-2 only stimulates proliferation of T-lymphocytes which express IL-2 receptor on their surfaces, OKT3 therapy will begin twenty-four hours prior to initiation of IL-2 administration (i.e., to activate T-lymphocytes to express IL-2 receptor). Following the initial intensificatioin regimen with hydroxyurea/ddI (supra) the stimulation of latently-infected cells with OKT3/IL-2 will lead to a profound decrease in clearance in persistently-infected cells, as compared to on-going studies of patients on HAART alone.

If hematological parameters (especially neutrophil count and CD4+ T-lymphocyte count) are not adversely affected by ddI and hydroxyurea (infra), the patients are brought into the Bone Marrow Transplant Unit and treated with a combination of OKT3 (Orthoclone OKT3, Ortho Biotech, Inc.) and IL-2 (Proleukin, Chiron, Corp.) to stimulate latently-infected cells with intensification therapy. ddI and hydroxyurea plus HAART is continued during OKT3/IL-2 stimulation therapy. OKT3 is administered intravenously (i.v.) at 400 micrograms on day one (given over 15 minutes). This is a lower dose than used for T-lymphocyte depletion, but will have T-lymphocyte activation effects, based on previously obtained data (supra). (Urba, W. J., et al., Cancer Res. 52: 2394-2401, 1992). This dose has been chosen to decrease side-effects when used in combination with IL-2 and to limit T-lymphocyte depletion acutely during therapy. No preparative treatment with corticosteroids is used at this dose, as corticosteroids have been shown to decrease the T-cell activation induced by OKT3 when given at milligram levels (Chatenoud, L., et al., Transplantation 49: 697-702, 1990. Chatenoud, L., et al., Transplantation 51: 334-338, 1991). In addition, subcutaneous IL-2, initially starting at 1.2 per m²×10⁶ I.U. subcutaneously qday, is administered on days 2 to 15. Decreases in IL-2 administration over this fourteen day cycle are based on clinical symptoms and are adjusted accordingly (i.e., based on effects of the cytokine release syndrome, infra). This dose of IL-2 is such that any potential adverse cytokine release effects are minimized (Khatri, V. P., et al., J. Clin. Invest. 101: 1378-1378, 1998; Losso, W., 6^{th} Conference on Retroviruses and Opportunistic Infections, Chicago, Ill., February 1999, #354). HAART and intensification therapy with ddI/hydroxyurea is continued during treatments with OKT3 and IL-2.

After treatment with OKT3/IL-2, the patients will continue on the ddI and hydroxyurea plus HAART. Three and fourteen days after treatment with OKT3 and IL-2, they will have proviral levels and replication-competent viral isolation analyzed from PBMCs and seminal cells.

Treatments with OKT3 and IL-2 are initiated with a physician present during a four to five day admission to the Bone Marrow Transplant Unit. The first dose of IL-2 is given 24 hours after infusion of OKT3, and continued for a total of fourteen days. After 72 hours, if the patient has no or mild adverse side-effects, then the patient can be discharged and followed in the Infectious Disease Clinic as an out-patient, continuing IL-2 administration at home. If CD4+ T-lymphocyte counts become and remain depressed after a cycle of OKT3 and IL-2, then, based on the Investigator's discretion, repeat cycles of solely IL-2 (with potentially higher doses) may be given in four to six week intervals, as IL-2 has been demonstrated alone to significantly increase CD4+ T-lymphocyte counts in HIV-1-infected-individuals (Davey, R. T., et al., J. Infect. Dis. 175: 781-789, 1997; Kovacs J. A., et al., N. Engl. J. Med. 332: 567-575, 1995; Kovacs, J. A., et al., N. Engl. J. Med. 335: 1350-1356, 1996). Of note, further treatments with OKT3 may not be possible due to the potential production of human anti-murine antibodies (HAMA) (Kreis, H., et al., Transplant Rev. 5: 181-199, 1991).

If replication-competent virus is no longer obtainable in these patients from any site after six months from the course of OKT3 and IL-2, then in discussion with the patient, stopping all anti-HIV-1 medications may be attempted with close monitoring to evaluate for potential “viral rebound”. This monitoring includes ultrasensitive HIV-1 RNA measurements monthly for one year, then every three months afterwards. Prior to stopping anti-virals, a tonsillar biopsy is obtained to evaluate potential HIV-1 replication and latency in lymphoid tissues. The sample is analyzed by in situ hybridization for HIV-1-specific RNA and cultured for replication-competent virus.

Primary Procedure

1) To determine the safety and tolerability of IL-2 and OKT3 as stimulation therapy for latently-infected cells with hydroxyurea and ddI as intensification therapy for patients on HAART, with undetectable plasma HIV-1 RNA.

2) Analyze the presence and levels of proviral DNA and replication-competent HIV-1 in PBMCs and seminal cells of these patients, over time, throughout therapies with IL-2/OKT3 and ddI/hydroxyurea.

Secondary Procedure

1) Determine if and when proviral sequences and replication-competent viruses from seminal and PBMC cells go below levels of detection. Then, in consultation with the patient, it is decided whether HAART is discontinued and the patient followed clinically and virologically.

2) Determine the in vivo half-life of proviral-containing cells and replication-competent virus levels in PBMCs and seminal cells in patients receiving the therapeutic regimen (supra) as compared to patients receiving HAART alone (non-study controls).

Selection and Enrollment of Subjects

Inclusion Criteria:

1) Age greater than 18 years old and the ability and willingness to give informed consent.

2) HIV-1 infection as documented by enzyme-linked immunosorbant assay (ELISA) and confirmed by Western Blotting.

3) CD4+ T-lymphocyte count greater than 200 cells/mm3.

4) HIV-1 RNA level in plasma less than 400 copies/ml for standard Roche RT-PCR and less than 500 copies/ml for branched chain DNA assays (Chiron) for at least one year, measured on at least two separate occasions. In addition, HIV1 plasma RNA level less than 50 copies/ml by ultrasensitive Roche RT-PCR at enrollment.

5) Stability on combination of anti-retroviral inhibitors that effectively decrease HIV-1 plasma RNA to below detectable levels by clinical ultrasensitive RT-PCR (limit 50 copies/ml). Numerous HAART combinations can be utilized for this effect. No change in HAART therapy over a one year period prior to the study is required.

6) Presence of HIV1 proviral DNA and replication-competent virus in PBMCs and/or seminal cells at enrollment and/or any detectable residual viral RNA in blood plasma (as determined by RT-PCR).

7) Normal chest x-ray 24 hours before therapy with Il-2/OKT3, and normal EKG.

8) Weight restricted to less than 3% above the patient's minimum weight during the week prior to initiating OKT3/IL-2 therapy.

Exclusion Criteria:

1) Due to overlapping bone marrow toxicity with hydroxyurea, Zidovudine (AZT) use in HAART is prohibited. In these patients, Zidovudine is switched to another RT inhibitor (but not ddI or D4T) for one month, for inclusion in this study.

2) Due to d4T's newly described additive, pancreatitis-inducing effects with ddI (ACTG 5025 and post-marketing reports to the FDA), d4T's (Stavudine) use in HAART is excluded. Patients on Stavudine must be switched to another RT inhibitor (not ddI or AZT) for one month for inclusion in this study.

3) Women will not be recruited into this study. Further studies are necessary to determine the persistent reservoirs in cervical/vaginal secretions. This is ongoing and, as such, women may be added to studies in the future.

4) At the investigator's discretion, any active substance abuse interfering with compliance.

5) Prior or present treatment with ddI, droxyurea, OKT3, or IL-2

6) Laboratory: Liver function tests (LFT's) greater than five times upper limit of normal; creatinine greater than 1.5 times upper limit of normal; ANC less than 1,500 cells per mm3; platelets less than 75,000; hemoglobin less than 8.8 grams per dL; lipase greater than two times upper limit of normal.

7) Subjects receiving acute or chronic therapy for cytomegalovirus (CMV), Mycobacterium avium intracellulare (MAC), toxoplasmosis, disseminated fungal infection or any significant medical illness as determined by the investigator.

8) Diagnosis of any malignancy.

9) Treatment with any candidate HIV-1 vaccine or immune modulating agent, including corticosteroids.

10) Transfusion with red blood cells within the past two months.

11) Treatment within the past two months with any colony-stimulating factor or erythropoietin, or with expectant need for these agents during the study.

12) Current Grade II or greater bilateral peripheral neuropathy.

13) History of pancreatitis or current alcohol abuse. 14) CD4+ T-lymphocyte count below 200 cells/mm3 at time of enrollment. The following exclusion criteria apply to the OKT3/IL-2 “stimulation” addition to the ddi/hydroxyurea therapeutic regimen:

15) History or presence of significant abnormalities of the central nervous system (CNS) (including meningitis), cardiovascular, pulmonary, renal, hepatic, or gastrointestinal systems.

16) Histroy or presence of autoimmune or inflammatory diseases.

17) History or presence of thyroid disease.

18) Exclude patients with known allergy to urine-derived products.

19) Exclude patients with history of seizures or who are predisposed to seizures.

20) Exclude patients with clinical signs of volume overload as evidenced by chest x-ray within 24 hours before the first dose of OKT3 or a greater than 3% weight gain in the week prior to OKT3 administration.

21) Exclude patients with congestive heart failure.

Enrollment Procedures:

Those subjects who fit criteria (supra) are screened with pre-entry evaluations. Referral is from HIV-1 treatment clinics.

As noted (supra), a CD4+ T-lymphocyte count greater than 200 cells/mm³ and a plasma viral RNA load less than 400 to 500 copies/ml, and then repeated with the ultrasensitive Roche RT-PCR showing less than 50 copies/ml, is required at the time of the initial screening for entry into the study. One month after the initial screening these tests are repeated to demonstrate that the CD4+ T-lymphocyte count remains above 200/mm³ and HIV-1 plasma viral RNA remains undetectable by ultrasensitive RT-PCR (less than 50 copies/ml).

A complete physical exam, especially noting any edema, including weight and assessment of peripheral neuropathy is obtained at screening. Safety laboratories include a complete blood count with differential and platelets. Chemistries will include electrolytes, glucose, BUN and creatinine, liver function studies including AST, ALT, alkaline phosphatase and total bilirubin, and lipase. Screening chest x-ray and EKG is also obtained on patients receiving OKT3/IL-2.

Randomization to the two study groups is via a computer-initiated random sequence generator.

Evaluations During Treatment

1) The following clinical and laboratory evaluations are performed at scheduled intervals. Weight at weeks: two, four, twelve, twenty-four, thirty-six, fifty-two, sixty-five, and seventy-eight.

2) Physical exam based on new and ongoing symptoms or signs at weeks, four, eight, twelve, twenty-four, thirty-six, fifty-two, sixty-five and seventy-eight.

3) Complete blood count (CBC) with differential and platelets at weeks: two, four, eight, twelve, sixteen, twenty-four, thirty-six, fifty-two, sixty-five, and seventy-eight.

4) Chemistries (supra) and liver function studies at weeks: two, four, eight, sixteen, twenty-four, thirty-six, fifty-two, sixty-five, and seventy-eight.

5) CD4 and CD8 T-lymphocyte counts (absolute and percentages) as performed by fluorescence-activated cell sorting (FACS) analyses at weeks: two, four, eight, twelve, eighteen, twenty-four, twenty-eight, thirty-six, forty-four, fifty-two, sixty-four, sixty-five and seventy-eight.

6) Following 4 weeks of therapy on OKT3/IL-2, quantitative HIV-1 RNA is determined by ultrasensitive assays (Roche RT-PCR). PBMCs and seminal cells for quantitative HIV-1 DNA -PCR to detect HIV-1 proviral levels, and subsequently co-cultured to detect replication-competent virus, are also determined at 4 weeks.

7) If these parameters (supra) are without significant alteration, as compared to baseline levels, (not including proviral DNA-PCR and replication-competent viral assays), then the patient is admitted to the Bone Marrow Transplant Unit for OKT3 and IL-2 therapy for a four to five day course. During this admission, the patient is monitored around the clock during therapy with OKT3 and IL-2.

8) During in-patient treatment with OKT3 and IL-2, daily CBC plus platelets and differential, electrolytes, BUN, creatinine, glucose, and LFTs are obtained.

9) On days two, four, and six ultrasensitive plasma HIV-1 RNA levels and CD4+ and CD8+ T-lymphocyte counts are obtained.

10) On days two, four and six of therapy with OKT3 and IL-2, cryopreserved PBMCs and plasma for HLA-DR T-lymphocyte positivity plus cytokines including TNF-α, IL-6, interferon gamma and IL-2 receptor levels (cell surface and soluble), are banked for analysis.

11) Aliquots of PBMCs and plasma are stored for further analyses, based on findings in the study as it develops. Of note, laboratory-based quantitative RT-PCR (sensitivity to 5 copies/ml) with confirmatory clinical ultrasensitive Roche RT-PCR (sensitivity to 50 copies/ml) is utilized in these studies.

12) During in-patient therapy with IL-2 and OKT3, no fewer than twice a day physical exams by a physician is performed, with chest x-rays obtained as needed. Vital signs are obtained q15 minutes during the first hour of therapy with OKT3 and then every 30 minutes for the next 2 hours, every hour for an additional 2 hours, and no fewer than q4 hrs during the individual's in-patient stay during the treatment cycle.

TABLE 2

| Time | Monitoring Parameters |
|---|---|
| First dose- | Vital signs every 15 minutes. |
| First hour | blood pressure |
| | temperature |
| | pulse |
| | respiration |
| First dose- | Vital signs every 30 minutes |
| | for the next 2 hrs. |
| | Every hour for an additional |
| | 2 hrs. |
| | Then, every 4 hrs. |
| For first few | Patients should remain under |
| Subsequent doses | close medical supervision. |
| | Vital signs every 30 minutes |
| | until stable. |

13) Upon discharge, the patient is seen within 3 days in the clinic for physical examination and repeat chemistries, LFTs, CBC plus differential and platelets, plasma viral RNA, and CD4+ T-lymphocyte counts. Crypopreserved PBMCs and plasma is stored for cytokine analysis and other T-lymphocyte subsets and proviral load and replication-competent viral isolation are obtained from PBMCs and serum.

14) The patient is seen in the clinic 7 days after discharge and then 14 days after discharge (off IL-2) and all lab evaluations (supra) are obtained.

15) The patients are evaluated in the clinic every two months and laboratory tests described (supra) are obtained. If, after six months, no replication-competent HIV-1 is found in PBMCs and seminal cells, then the patient is offered a trial off all anti-viral therapy.

16) Human anti-mouse antibody (HAMA) levels are obtained four weeks after OKT3 therapy.

17) TSH evaluations at weeks (post-IL-2/OKT3) six, eighteen and thirty.

Administration of Drugs

Orthoclone OKT3

Orthoclone OKT3 is supplied in 5 mg (1 mg/ml) vials and is a clear colorless solution. Vials are used only once. Prepare ORTHOCLONE OKT3 for injection by drawing solution into a syringe through a low-protein binding 0.2 or a 0.22 mm (micrometer) filter. After withdrawal, discard the filter and attach a new needle for intravenous injection. The appropriate dose of OKT3 is diluted in 15 ml of sterile normal saline for bolus injections over 15 minutes.

IL-2 (Proleukin)

IL-2 (Proleukin) is supplied in single use vials of 22×10⁶ IU (1.3 mg). Reconstituted or diluted Proleukin is stable for up to 48 hours at refrigerated temperatures and is stored in a refrigerator.

The lyophilized power is suitable for subcutaneous injection after reconstitution with 1.2 ml of sterile water and dilution with 5% Dextrose injection.

During reconstitution, the sterile water is directed at the side of the vial and swirled gently to avoid excess foaming without shaking. 5% dextrose injection, USP, is used to dilute the reconstituted solution. Tuberculin syringes are used to administer the doses of IL-2 (Proleukin) subcutaneously.

Virology

Quantitative HIV-1 RNA by ultrasensitive assays at weeks: two, four, eight, twelve, eighteen, twenty-four, twenty-eight, thirty-six, forty-four, fifty-two, sixty-five and seventy-eight. Laboratory-based quantitative RT-PCR on each date (sensitivity to 5 copies/ml), with confirmatory clinical ultrasensitive Roche RT-PCR (sensitivity to 50 copies/ml) at weeks 4, 8, 12, 18, 28, 36, 52 and 78.

Peripheral blood mononuclear cells (PBMCs) and seminal cells for DNA-PCR to detect HIV-1 proviral levels and co-culture to detect replication-competent virus at weeks: zero (baseline), eight, eighteen, twenty-four, thirty-six, forty-four, fifty-two, sixty-five and seventy-eight.

Proviral Reservoir(s) Decay and Interim Analysis

As the HIV-1 proviral reservoirs may have a significantly long T_{½} in vivo, an interim analysis of patients is conducted after one year of therapy (infra). Half of the ddI/hydroxyurea treated patients are randomized in a blinded fashion after one year of therapy. If no changes in PBMC or seminal cell reservoirs or residual plasma viral RNA are demonstrated in this sub-group, then this study is discontinued.

General Toxicity

OKT3

The endogenous release of cytokines from activated lymphocytes or monocytes is often demonstrated with administration of OKT3 at doses of microgram to milligram levels. This cytokine release syndrome (CRS) can lead to side-effects that include, but are not limited to, fever, chills, headache, vomiting, diarrhea, tachycardia, hypotension, bronchospasm and arthralgia. The frequency and severity of CRS is usually greatest after the first dose of OKT3, with each successive dose leading to decreased severity and frequency of CRS. CRS ranges from a more frequently reported mild, self-limited, flu-like illness, to less frequently reported severe, life-threatening, shock-like reactions which may include serious cardiovascular and CNS manifestations. Other adverse experiences occurring in 8% or more of the patients during the first two days of ORTHOCLONE OKT3 therapy include: dyspnea (21%), nausea (19%), vomiting (19%), chest pain (14%), tremor (13%), wheezing (13%), headache (11%), tachycardia (10%), rigor (8%), and hypertension (8%). In addition, seizures, encephalopathy, cerebral edema, aseptic meningitis and headache, have been reported during treatment with ORTHOCLONE OKT3.

Anaphylactic or anaphylactoid reactions may occur following any administration of any dose or course of ORTHOCLONE OKT3. Of importance, the anaphylactic reactions which can occur with OKT3, usually occur within ten minutes of injection, while signs and symptoms that mimic this 30 to 60 minutes or later after infusion are usually due to CRS (Kreis, H., et al., Transplant Rev. 5: 181-199, 1991; Abramowica, D., et al., Transplantation 47: 606-608, 1989; Chatenoud, L., et al., Transplantation 49: 697-702, 1990, Chatenoud, L., et al., Transplantation 51: 334-338, 1991; Chatenoud, L., et al., N. Engl. J. Med. 320: 1420-1421, 1989; Ferran, C., et al., Eur. J. Immunol. 20: 509-515, 1990). Serious and occasionally life-threatening systemic, cardiovascular, and central nervous system reactions have been reported following administration of ORTHOCLONE OKT3. These have included: pulmonary edema, especially in patients with volume overload; shock; cardiovascular collapse; cardiac or respiratory arrest; seizures and coma.

As noted (supra), an aseptic meningitis syndrome characterized by fever, headache, neck stiffness and photophobia can be seen after treatment with OKT3. This diagnosis is confirmed by demonstrating increased protein and white blood cells in the CSF with negative bacterial cultures. In studies by Bluestone, et al., significantly lower side effects were noted with humanized anti-T3 antibody administered at 200-400 μs, as compared to milligram doses.

In addition, treatment with high dose OKT3 has yielded increased risk for lymphoproliferative disorders, lymphomas and skin cancers. These are believed to be due to suppression of cytotoxic T-lymphocyte allowing proliferation and transformation of EBV-infected B-lymphocytes. Of importance, the lymphoproliferative disorders associated with EBV may regress if detected early, thus leading to discontinuation of OKT3 treatments (Kreis, H., et al., Transplant Rev. 5: 181-199, 1991).

Interleukin-2

IL-2 therapy has been associated with a capillary leak syndrome (CLS), which is manifested by decreased vascular tone and extravastation of plasma protein and fluid into extravascular space. This can result in hypotension and may be so severe as to lead to reduced organ perfusion and death. CLS has also been associated with cardiac arrhythmias, angina, myocardial infarction, respiratory insufficiency, gastrointestinal bleeding and infarction, renal insufficiency, edema and changes in cognition (Siegla, J. P., Puri, R. K. J. Clin. Oncol. 9: 694, 1991). CLS is more common in patients with underlying cardiac disease and fluid overload. CLS appears to be significantly less common in patients treated with subcutaneous rather than intravenous. People treated with IL-2 at high doses have shown to have impaired polymorphonuclear leukocyte function with increased risk of severe infections. As such, there are data to suggest that during high dose IL-2 therapy, patients with indwelling central venous catheters should be treated with antibiotics prophylactially to inhibit staphylococci (Bock, S. N., et al., J. Clin. Oncol. 8: 161-169, 1990; Hartman, L. C., et al., J. Natl. Cancer Inst. 81: 1190-1193, 1989). High dose IL-2 therapy has been demonstrated to lead to severe central nervous system effects including lethargy, somolence and rarely coma. CLS usually begins immediately after high dose IL2 therapy. Of note, a delayed allergic reaction to iodinzated contrast media may occur after IL-2 therapy (Choyke, P. L., et al., Radiology 183: 111-114, 1992).

IL-2 therapy has been associated with exacerbation of pre-existing or initial presentation of autoimmune disease and inflammatory disorders. Exacerbation of Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid has been reported following treatment with IL-2 (Siegla, J. P., Puri, R. K. J. Clin. Oncol. 9: 694, 1991).

Toxicity Management

The NIAID standardized toxicity grading system will be utilized (Table 3). Toxicity is graded on a 1 to 4 scale. When toxicity is encountered the dose administered to subjects is modified. Level 1 dose reduction for ddI is to 250 mg po qday (or 175 mg po qday for patients less than 60 kgs). Level 2 dose reduction will include discontinuation from the study. For hydroxyurea, Level 1 dose reduction will be to 800 mg qday (400 mg po BID). Level 2 dose reduction will be to 600 mg po qday (300 mg po BID), with a Level 3 reduction leading to discontinuation from the study.

Study drug modifications that are likely to be secondary to one of the two medications is performed with one drug at a time, starting with the drug that is most likely to have caused the toxicity. Of note, hydroxyurea is mainly associated with anemia, neutropenia, and thrombocytopenia. Pancreatitis, peripheral neuropathy, nausea, vomiting, and diarrhea are associated with ddI. For Grade 1 toxicity, there are no dose modifications or interruptions. For Grade 2 toxicity, except for hematological toxicities, there are no dose modifications. Grade 3 or greater (or an increase in toxicity over two grades for hematological): dose reductions and modifications are made for all Grade 3 and 4 toxicities, or for an increase in toxicity over two grades for hematological toxicity, judged to be drug-related.

Laboratory values indicating Grade 3 or Grade 4 toxicity that are inconsistent with previous values are confirmed immediately. If the study medication must be interrupted for greater than 28 days the patient will not continue in this study.

Toxicities determined or suspected to be not related to study drugs are managed through best clinical practice.

These parameters are not used during acute OKT3/IL-2 treatment (defined as the week of therapy and one week after). Unremitting Grade 3 or 4 neutropenia is an exception, and hydroxyurea may be interrupted during the OKT3/IL-2 treatment period based on the best clinical judgement of the Investigators.

If the CD4+ T-lymphocyte count is below 50% of baseline level, then hydroxyurea may be withheld until the level recovers to this level.

TABLE 2

| | | | | GRADE 4 |
|---|---|---|---|---|
| | | | | POTENTIALLY |
| | GRADE 1 | GRADE 2 | GRADE 3 | LIFE |
| PARAMETER | MILD | MODERATE | SEVERE | THREATENING |
| HEMATOLOGY | | | | |
| Hemoglobin | 8.0 g/dL-9.4 g/dL | 7.0 g/dL-7.9 g/dL | 6.5 g/dL-6.9 | <6/5 g/dL |
| | | | g/dL | |
| Absolute | 1000-1500/mm³ | 750-999/mm³ | 500-749/mm³ | <500/mm³ |
| Neutrophil Count | | | | |
| Platelets | 75.000-99.000/mm³ | 50.000-74.999/ | 20.000-49,999/ | <20.000/mm³ |
| | | mm³ | mm³ | |
| Prothrombin | >1.0-1.25 × ULN | >1.25-1.5 × ULN | >1.5-3.0 × ULN | >3 × ULN |
| Time (PT) | | | | |
| PTT | >1.0-1.66 × ULN | >1.66-2.33 × ULN | >2.33-3.0 × ULN | >3.0 × ULN |
| Methemoglobin | 5.0-10.0% | 10.1-15.0% | 15.1-20.0% | >20% |
| CHEMISTRIES | | | | |
| SODIUM | | | | |
| Hyponatremia | 130-135 me/L | 123-129 meq/L | 116-122 meq/L | >116 me/L |
| Hypernatremia | 146-150 meq/L | 151-157 meq/L | 158-165 meq/L | >165 meq/L |
| POTASSIUM | | | | |
| Hypokalemia | 3.0-3.4 meq/L | 2.5-2.9 meq/L | 2.0-2.4 meq/L | <2.0 meq/L |
| Hyperkalemia | 5.6-6.0 meq/L | 6.1-6.5 meq/L | 6.6-7.0 meq/L | >7.0 meq/L |
| PHOSPHATE | | | | |
| Hypophosphatemia | 2.0-2.4 mg/dL | 1.5-1.9 mg/dL | 1.0-1.4 mg/d | <1.0 mg/dL |
| CALCIUM | | | | |
| (corrected for albumin) | | | | |
| Hypocalcemia | 7.8-8.4 mg/dL | 7.0-7.7 mg/dL | 6.1-6.9 mg/dL | <6.1 mg/dL |
| Hypercalcemia | 10.6-11.5 mg/dL | 11.6-12.5 mg/dL | 12.6-13.5 m/dL | >13.5 mg/dL |
| MAGNESIUM | | | | |
| Hypomagnesemia | 1.2-1.4 meq/L | 0.9-1.1 meq/L | 0.6-0.8 meq/L | <0.6 meq/L |
| BILIRUIBIN | | | | |
| Hyperbilirubinemia | >1.0-1.5 × ULN | >1.5-2.5 × ULN | >2.5-5 × ULN | <5 × ULN |
| GLUCOSE | | | | |
| Hypoglycemia | 55-64 mg/dL | 40-54 mg/dL | 30-39 mg/dL | <30 mg/dL |
| Hyperglycemia | 116-160 mg/dL | 161-250 mg/dL | 251-500 mg/dL | >500 mg/dL |
| (Nonfasting and no prior diabetes) | | | | |
| Triglycerides | | 400-750 mg/dL | 751-1200 mg/dL | >1200 mg/dL |
| Creatinine | >1.0-1.5 × ULN | >1.5-3.0 × ULN | >3.0-6.0 × ULN | >6.0 × ULN |
| URIC ACID | | | | |
| Hyperuricemia | 7.5-10.0 mg/dL | 10.1-12.0 mg/dL | 12.1-15.0 mg/dL | >15.0 mg/dL |
| LIVER TRANSAMINASE (LFTs) | | | | |
| AST (SGOT) | 1.25-2.5 × ULN | >2.5-5.0 × ULN | >5.0-10.0 × ULN | >10.0 × ULN |
| ALT (SGPT) | 1.25-2.5 × ULN | >2.5-5.0 × ULN | >5.0-10.0 × ULN | >10.0 × ULN |
| GGT | 1.25-2.5 × ULN | >2.5-5.0 × ULN | >5.0-10.0 × ULN | >10.0 × ULN |
| Alk Phos | 1.25-2.5 × ULN | >2.5-5.0 × ULN | >5.0-10.0 × ULN | >10.0 × ULN |
| PANCREATIC ENZYMES | | | | |
| Amylase | >1.0-1.5 × ULN | >1.5-2.0 × ULN | >2.0-5.0 × ULN | >5.0 × ULN |
| Pancreatic | >1.0-1.5 × ULN | >1.5-2.0 × ULN | >2.0-5.0 × ULN | >5.0 × ULN |
| amylase | | | | |
| Lipase | >1.0-1.5 × ULN | >1.5-2.0 × ULN | >2.0-5.0 × ULN | >5.0 × ULN |
| CARDIOVASCULAR | | | | |
| Cardiac | | Asymptomatic: | Recurrent/persistent | Unstable |
| Arrhythmia | | transient | dysrhytbmia: | dysrhythmia. |
| | | dysrhythmia, no RX | Symptomatic. RX | Hospitalization |
| | | required. | required. | and |
| | | | | RX required. |
| Hypotension | Transient orthostatic | Symptoms | IV fluid req. No | Hospitalization |
| | Hypotension. No RX. | correctable with oral | hospitalization | required. |
| | | fluid RX. | required. | |
| Hypertension | Transient. Increase | Recurrent: chronic | Acute Rx required: | Hospitalization |
| | >20 mm/Hg: no RX. | increase >20 | outpatient | required. |
| | | mm/Hg. Rx | hospitalization | |
| | | required. | possible. | |
| Pericarditis | Minimal effusion. | Mild/mod | Symptomatic | Tamponade OR |
| | | asymptomatic | effusion. Pain. | pericardiocenteisis |
| | | effusion. No Rx. | EKG changes. | OR surgery |
| | | | | required. |
| Hemorrhage, | | Mildly symptomatic. | Gross blood loss | Massive blood |
| Blood Loss | | No Rx required. | OR 1-2 units | loss OR >2 units |
| | | | transfused. | transfused. |
| GASTROINTESTINAL | | | | |
| Nausea | Mild OR transient: | Mod discomfort OR | Severe discomfort | Hospitalization |
| | reasonable intake | intake decreased for | OR minimal intake | required. |
| | maintained | <3 days | for •3 days. | |
| Vomiting | Mild OR transient 2-3 | Mod OR persistent: | Severe vomiting of | Hypotensive |
| | episodes per day OR | 4-5 episodes per | all food/fluids in | Shock OR |
| | mild vomiting lasting | day: OR vomiting | 24 hours OR | hospitalization |
| | <1 week. | lasting •1 week. | orthostatic | required for IV RX |
| | | | hypotension OR IV | required. |
| | | | RX required. | |
| Diarrhea | Mild OR transient: 3-4 | Mod OR persistent: | Bloody diarrhea: | Hypotensive |
| | loose stools per day OR | 5-7 loose stools per | OR orthostatic | shock OR |
| | mild diarrhea lasting <1 | day OR diarrhea | hypotension OR >7 | hospitalization |
| | week, | lasting •1 week, | loose stools/day | required. |
| | | | OR IV RX | |
| | | | required. | |
| Oral | Mild discomfort. No | Difficulty | Unable to swallow | Unable to drink |
| Discomfort/Dysp | difficulty swallowing. | swallowing but able | solids. | fluids: IV fluids |
| hagia | | to eat and drink, | | required. |
| Constipation | Mild. | Moderate. | Severe. | Distention with |
| | | | | vomiting. |
| RESPIRATORY | | | | |
| Cough (for | Transient: no Rx. | Treatment | Uncontrolled | |
| aerosol studies) | | associated cough: | cough: systemic RX | |
| | | inhaled | required. | |
| | | bronchodilator. | | |
| Bronchospasm | Transient: no RX: FEV1 | RX required: | No normalization | Cyanosis: FEV 1 |
| Acute | 70%-<80% (or peak | normalizes with | with | <25% (or peak |
| | flow). | bronchodilator: | bronchodilator: | flow) OR |
| | | FEV1 50%-<70% (or | FEV1 25%-<50% | intubated. |
| | | peak flow) | (or peak flow). | |
| | | | Retractions. | |
| Dyspnea | Dyspnea on exertion. | Dyspnea with | Dyspnea at rest. | Dyspnea |
| | | normal activity. | | requiring O₂ |
| | | | | therapy. |
| NEUROLOGIC | | | | |
| Neuro- | Slight incoordination | Intention tremor OR | Ataxia requiring | Unable to stand. |
| cerebellar | OR | dysmetria OR | assistance to walk | |
| | dysdiadochokinesia. | slurred speech OR | or arm | |
| | | nystagmus. | incoordination | |
| | | | interfering with | |
| | | | ADLS. | |
| Neuro- | | | Severe mood | Acute psychosis |
| psych/mood | | | changes requiring | requiring |
| | | | medical | hospitalization. |
| | | | intervention. | |
| Paresthesia | Mild discomfort: no RX | Mod discomfort: | Sever discomfort: | Incapacitating: |
| (burning or | required. | non-narcotic | OR narcotic | OR not responsive |
| tingling, etc.) | | analgesia required. | analgesia required | to narcotic |
| | | | with symptomatic | analgesia. |
| | | | improvement. | |
| Neuro-motor | Mild weakness in | Mod weakness in | Marked distal | Confined to bed |
| | muscle of feet but able | feet (unable to walk | weakness (unable | or wheel chair |
| | to walk and/or mild | on heels and/or | to dorsiflex toes or | because of muscle |
| | increase or decrease in | toes). Mild | foot drop) and | weakness. |
| | reflexes. | weakness in hands, | mod proximal | |
| | | Still able to do most | weakness, e.g., in | |
| | | hand tasks and/or | hands interfering | |
| | | loss of previously | with ADLs and/or | |
| | | present reflex or | requiring | |
| | | development of | assistance to walk | |
| | | hyper-reflexia | and/or unable to | |
| | | and/or unable to do | rise from chair | |
| | | deep knee bends | unassisted | |
| | | due to weakness. | | |
| Neuro-sensory | Mild impairment (dec | Mod impairment | Severe impairment | Sensory loss |
| | sensation, e.g., | (mod dec sensation, | (dec or loss of | involves limbs |
| | vibratory, pinprick, | e.g., vibratory | sensation to knees | and trunks. |
| | hot/cold in great toes) | pinprick, hot/cold | or wrists) or loss of | |
| | focal area or | to ankles) and/or | sensation of at least | |
| | symmetrical | joint position or | mod degree in | |
| | distribution, | mild impairment | multiple different | |
| | | that is not | body areas (i.e., | |
| | | symmetrical | upper and lower | |
| | | | extremities) | |
| URINALYSIS | | | | |
| Proteinuria | | | | |
| Spot urine | 1+ | 2-3+ | 4+ | Nephrotic |
| | | | | syndrome |
| 24-hour urine | 200 mg-1 g loss/day OR | >1-2g loss/day OR | >2.0-3.5 gloss/day | Nephrotic |
| | <0.3% OR <3 g/L. | 0.3-1.0% OR >10 | OR >1.0% OR >10 | syndrome OR |
| | | g/L. | g/L. | >3.5 gloss/day. |
| Gross Hematuria | Microscopic only | Gross. No dots. | Gross plus clots. | Obstructive OR |
| | | | | transfusion |
| | | | | required. |
| MISCELLANEOUS | | | | |
| Fever | 37.7-38.5 C OR | 38.6-39.5 C OR | 39.6-40.5 C OR | >40.5 C OR |
| Oral >12 | 100.0-101.5 F | 101.6-102.9 F | 103-105 F | >105 F |
| hours | | | | |
| Headache | Mild: no RX required. | Mod: or non | Severe: OR | Intractable: OR |
| | | narcotic analgesia | responds to initial | requiring repeated |
| | | RX. | narcotic RX. | narcotic RX. |
| Allergic | Pruritus without rash. | Localized urticaria. | Generalized | Analyphylaxis. |
| Reaction | | | urticaria | |
| | | | angloedema. | |
| Cutaneous/Rash/ | Erythema. | Diffuse | Vesiculation OR | ANY ONE: |
| Dermatitis | | maculopapular rash | moist | mucous |
| | | OR dry | desquamation OR | membrane |
| | | desquamation. | ulceration. | involvement. |
| | | | | Suspected |
| | | | | Stevens-Johnson |
| | | | | (TEN). Erythema |
| | | | | multiforme. |
| | | | | Necrosis requiring |
| | | | | surgery. |
| | | | | Exfoliative |
| | | | | dermatitis. |
| Local Reaction- | Erythema. | Induration <10 mm | Induration >10 mm | Necrosis of skin. |
| Parenteral Rx | | OR inflammation | OR ulceration. | |
| No medication or | | OR phlebitis. | | |
| other | | | | |
| Activity Scale | Normal activity | Normal activity | Normal activity | Unable to care for |
| | reduced 25%. | reduced 25-50%. | reduced >50%: | self. |
| | | | cannot work. | |

Abbreviations used in Table 3

“ULN” means “upper limit of normal”

“Rx” means “therapy”

“Mod” means “moderate”

“ADL” means “activities of daily living”

“LLN” means “lower limit of normal”

“Req” means “required”

“IV” means “intravenous”

“Dec” means “decreased”

Estimating Severity Grade

For abnormalities NOT found elsewhere in Table 3, use the scale below to estimate grade of severity:

GRADE 1: Mild Transient or mild discomfort. No limitation in activity. No medical intervention/therapy required.

GRADE 2: Moderate Mild to moderate limitation in activity-some assistance may be needed. No minimal medical Intervention/therapy required.

GRADE 3: Severe Marked limitation in activity. Some assistance usually required. Medical intervention/therapyrequired. Hospitalizations possible.

GRADE4: Life-Extreme limitation in activity. Significant assistance required. Significant medical intervention/threatening therapy required. Hospitalization or hospice care probable.

Serious or Life-threatening Adverse Experiences

ANY clinical event deemed by the clinician to be serious or life-threatening should be considered a grade 4 adverse experience. Clinical events considered to be serious or life-threatening include but are not limited to: seizures, coma, tetany, diabetic ketoacidosis, disseminated intravascular coagulation, diffuse petechia, paralysis, acute psychosis.

when two values are used to define the criteria for each parameter, the lowest values will be first.

Parameters are generally grouped by body system.

some protocols may have additional protocol specific grading criteria.

IL-2 and OKT3

1) IL-2 is considered for permanent discontinuation, and the patient removed from the study, if Grade 4 toxicities (from IL-2 and/or OKT3) occur.

2) Three dose reductions will be utilized for IL-2:

0.6×10⁶ IU per m² sq QD

0.3×10⁶ IU per m² sq QD

Discontinued

3) Toxicities to cause IL-2 interruption:

a) Grade 2 or worse:

sodium and potassium

respiratory (evaluate carefully for CLS)

neurological

b) Grade 3 of any toxicity not mentioned (supra). (Note: any edema will lead to evaluation for CLS). See Standard Division of AIDS Toxicity Chart in Table 3.

IL-2 Dose Modifications

1) For any toxicities which lead to IL-2 interruption, this therapy will be stopped until toxicity is one grade below the grade which led to treatment interruption.

2) Then, the patient is started on IL-2 at Dose Reduction 1.

3) If the patient has the same toxicities, as occurred originally, IL-2 is interrupted until toxicity resolves. Then, IL-2 is restarted at Dose Reduction 2.

4) If the patient again has the toxicities at Dose Reduction 2, then study treatments are permanently discontinued, and the patient followed carefully until toxicities resolve.

5) IL-2 administrations will be withheld until any Grade 3 toxicities secondary to OKT3 returns to Grade 2 or lower.

Bilateral Peripheral Neuropathy

The Grade of bilateral peripheral neuropathy (defined in Table 3) to be used for purposes of dose modification is the maximum of the grades of paresthesia, neuro-motor, and neuro-sensory toxicities.

For greater than Grade 3 bilateral peripheral neuropathy, ddI is discontinued until the bilateral peripheral neuropathy returns to less than Grade 2. The subject is then re-challenged at level one dose reduction. For subjects whose bilateral neuropathy does not improve to less than Grade 2 after 28 days or those with a recurring drug-related bilateral peripheral neuropathy of greater than Grade 3, ddI is permanently discontinued. If no recurrence occurs while on the level of one dose reduction, the subject may be re-challenged at the original dose.

For a Grade 4 bilateral peripheral neuropathy, the subject will not continue in the study and ddI is discontinued.

Hematologic

For greater than Grade 2 anemia or thrombocytopenia judged to be study drug related, hydroxyurea is withheld until the toxicity returns to Grade 1 or less, then restarted at a level of one dose reduction. If the greater than Grade 2 toxicity recurs, the hydroxyurea is withheld until the toxicity returns to Grade 1 or less, then restarted at a level of two dose reduction. If the toxicity does not resolve within 28 days on the dose reduction or if greater than Grade 2 toxicity recurs on the level two reduction, hydroxyurea is permanently discontinued.

For greater than Grade 2 anemia judged to be study drug related, subjects are treated with blood transfusions and/or erythropoietin therapy.

For greater than Grade 3 neutropenia, hydroxyurea is withheld until the toxicity returns to Grade 1 or less, then restarted at a level of one dose reduction. If the greater than Grade 2 toxicity recurs, the hydroxyurea is withheld until the toxicity returns to Grade 1 or less then restarted at a level of two dose reduction. If the toxicity does not resolve within 28 days on the dose reduction or if greater than Grade 2 toxicity recurs on the level two reduction, hydroxyurea is permanently discontinued. For greater than Grade 3 neutropenia, granulocyte colony-stimulating factor (G-CSF) may be instituted for toxicity management while hydroxyurea is withheld. For Grade 4 neutropenia, G-CSF is strongly encouraged until the neutropenia is Grade 1 or less. G-CSF is not administered within 72 hours of receiving hydroxyurea.

For hematologic toxicities thought to be secondary to hydroxyurea that require one or more dose reductions, no attempt are made to increase the dose of hydroxyurea back to full dose.

Hyper-lipasemia

If greater than Grade 1, obtain a repeat test. If the test is normal continue ddI, if abnormal (or unavailable in 24 hours) hold ddI until less than Grade 2 toxicity or baseline (continue dosing hydroxyurea), then restart at a level one reduction. If toxicity recurs, discontinue ddI. If no recurrence occurs, subject may be re-challenged at original ddI dose.

If Grade 3 or 4 hyper-lipasemia persists longer than two weeks while ddI is withheld, ddDI is discontinued and hydroxyurea is withheld until lipase is less than Grade 2.

Nausea/Vomiting

If greater than Grade 3 nausea and/or vomiting, ddI is withheld until the toxicity returns to less than Grade 2, dosing with hydroxyurea is continued. When the toxicity returns to less than Grade 2, study medication is resumed at a level one reduction. If the toxicity does not resolve within 7 days, hydroxyurea is withheld. When the toxicity returns to less than Grade 2, resume hydroxyurea first at a level one reduction followed one week later by ddI at a level one reduction. If the toxicity still does not resolve within 28 days, all study medications are permanently discontinued.

Of note, substitution of ddI tablets for the pediatic powder for those patients with gastrointestinal intolerance can be utilized.

Pancreatitis

Grade 3 nausea, vomiting or abdominal pain associated with a Grade 2 elevation of serum lipase is classified as pancreatitis: discontinue ddI permanently and hold hydroxyurea. Resume hydroxyurea when nausea, vomiting or abdominal pain is less than Grade 2. If Grade 3 symptoms of pancreatitis persist, hydroxyurea is discontinued.

Other Grade 3 or Grade 4 Toxicities

For all other Grade 3 or greater toxicities judged to be study drug related, all study drugs are withheld until the toxicity returns to less than Grade 2.

In situations where both drugs are being withheld due to toxicity and the toxicity resolves to a Grade 2 or less, then the drugs are re-introduced at the appropriate dose reduction level, one at a time, beginning with the drug that, in the opinion of the Investigator, is more likely to have caused the toxicity. The second drug is resumed one week after the first agent.

For other toxicities that require dose reduction, an attempt at increasing the dose of the medication back to full dose is encouraged at the discretion of the Investigator. Factors that are considered include: 1) the type of toxicity; 2) the duration of the toxicity; and 3) the likelihood that a concomitant medication or illness which is stopped then restarted at a level one dose reduction. If the greater than Grade 2 toxicity recurs, the hydroxyurea is withheld until the toxicity returns to less than Grade 1, then restarted at a level two dose reduction. If the toxicity does not resolve within 28 days on the dose reduction or if greater than Grade 2 toxicity recurs on the level two reduction, hydroxyurea is permanently discontinued.

Criteria for Treatment Discontinuation:

1) The subject refuses further treatment and/or follow-up evaluations.

2) The Investigator determines that further participation is detrimental to the subject s health or well-being.

3) The subject fails to comply with the study requirements so as to cause harm to self or seriously interfere with the validity of the study results.

4) The subject requires treatment with medications which are disallowed while on this study.

5) Drug toxicity (supra).

6) Discontinuation of study by IRB, FDA, and/or Bristol-Myers Squibb.

7) Virologic failure.

8) Interim study results mandate discontinuation.

9) Evidence of anaphylacitc reactions.

10) Evidence of capillary leak syndrome (CLS).

Concomitant Medications (Allowed and Disallowed)

Disallowed or Restricted

1) Requirement for additional (or switching) anti-retroviral therapies during the study to maintain undetectable viral RNA in blood plasma.

2) Systemic chemotherapy for active malignancies including systemic therapy for Kaposi's Sarcoma (KS) is not permitted.

3) Other agents with myelosupressive potential including tegretol, carboplatin, carmustine, cyclophosphamide and fluorouracil are restricted or avoided whenever possible during treatment on this study.

4) Granulocyte colony stimulating factor (G-CSF) is not permitted except during toxicity management while hydroxyurea is withheld. G-CSF is given only after 72 hours have elapsed since the last dose of hydroxyurea.

5) Drugs associated with peripheral neuropathy (other than ddI) are restricted or avoided wherever possible during treatment on this study. These drugs include, but are not limited to: hydralazine, disulfiram, nitrofurantoin, cisplatinum, diethyldithiocarbamate gold, rifampin, chloramphenicol, clioquinol, ethambutol, ethionamide, glutethimide, sodium cyanate and thalidomide.

6) Corticosteroids are only allowable for treatment of severe and unremitting CRS.

7) Agents which independently alter the CNS, including sedatives and anti-seizure medications.

8) Iodinated contrast dyes.

9) Other cardiotoxic agents.

10) Interferon alpha

11) Other potential hepatotoxic or renal toxic agents, excluding anti-retrovirals.

12) Anti-hypertensive agents, including beta blockers.

13) Indomethicin.

14)Aspirin

Drugs associated with known or suspected toxicity of the pancreas are restricted or avoided wherever possible while patients are taking ddI, including I.V. pentamidine and ethyl alcohol. ddI is temporarily dose-interrupted for Pneumocystis carinii pneumonia (PCP) therapy with I.V. pentamidine. The package inserts are consulted to determine contraindicated concomitant medications.

Allowed

PCP prophylaxis with trimethoprim/sulfamethoxazole (Bactrim, Septra) or Dapsone is allowed and is used at the discretion of the Investigator when clinically indicated.

Anti-emetics, diphenylhydramine, acetaminophen, and anti-diarrheals are allowed during treatment with OKT3 and IL-2, in addition to during intervening periods.

Low doses of meperidine and non-steroidal anti-inflammatory drugs (except for indomethicin) for CRS during treatment with OKT3.

Vasopresors (especially dopamine) can be utilized for severe hypotension.

Records to be Kept

Case Report Forms (CRF) are provided for each subject. Subjects must not be identified by the name on any study documents. Subjects are identified by the Patient Identification Number (PID).

Statistical Considerations

The clearance of the persistently HIV-1 infected cell population(s) is assumed to occur based upon first order kinetics. Therefore, any time-point will serve as the baseline, or reference time-point, and the baseline need not be considered as the day anti-retroviral therapy is initiated. Kinetic modeling will assume a single compartment model with underlying normality of kinetic parameters and a baseline assumption of a half-life of 350 days and 60 day inter-patient variability. Using the formula clearance=e^{-kt}, based upon the above assumptions, k=0.0028+/−0.00049. Table 4 indicates the sample sizes to have 80% and 90% power to detect the above slope with a 10% or 20% error.

TABLE 4
Subjects required

| | | |
|---|---|---|
| Power | 80% | 90% |
| Error | | |
| 10% | 13 | 15 |
| 20% | 4 | 5 |

Therefore, assuming a drop-out rate of 10% and that 10% of subjects may experience a rebound in viral load during the follow-up period, there is adequate power to estimate the kinetics of clearance of the persistently-infected cell population with twenty-four participants. This model holds under either of the following two assumptions: 1) the system is in steady state, that is, the model assumes that the persistently infected cells in all compartments are rapidly transferring back and forth so that the depletion of persistently infected cells from the genital secretions reflects the depletion of persistently infected cells in the entire body; or 2) no transfer of cells is occurring from other pools of infected cells and therefore the system is closed. With respect to semen, either assumption is operative.

Comparisons of clearance among hydroxyurea/ddI and non-hydroxyurea/ddI recipients are done by the following analyses. With eight subjects in each group (standard anti-retroviral therapy recipients and standard anti-retroviral therapy recipients plus hydroxyurea and ddI -16 total), if the half-life is 350 days in those treated with standard anti-retroviral therapy, a study may have 80% power to be able to detect a half-life in the ddI/hydroxyurea-treated group of 285 days. With 6 subjects (3 in each group), if the half-life is 350 days in those treated with standard anti-retroviral therapy, a study may have 80% power to be able to detect a half-life in the ddI/hydroxyurea-treated group of 250 days. If we recruit 12 subjects on standard anti-retroviral therapy and 12 subjects on standard anti-retroviral therapy plus ddI and hydroxyurea, adequate power to discern differences in clearance rates between the two populations is obtained. Nevertheless, these are gross estimates, as little data on the in vivo T_{½} of HIV-1 proviral species is available (Zhang, H., et al., N. Engl. J. Med. 339(25): 1803-1809, 1998; Dornadula, G., et al., JAMA 282(17): 1627-1632, 1999).

During the OKT3/IL-2 therapeutic regimen, if this approximation is correct for “non-stimulated” cells, a larger study will be required.

Human Subjects

Institutional Review Board (IRB) Review and Informed Consent

This protocol and the informed consent document and any subsequent modifications are reviewed and approved by the Institutional Review Board for oversight of the study. Written informed consent is obtained from the subjects. The subjects' assent must also be obtained if he is able to understand the nature, significance and risks associated with the study. The informed consent will describe the purpose of the study, the procedures to be followed and the risks and benefits of participation. A copy of the consent form is given to the subject.

Subject Confidentiality

All laboratory specimens, evaluation forms, reports, and other records are identified by a coded number only to maintain subject confidentiality. All records are kept in a locked file cabinet. All computer entry and networking programs are performed with coded numbers only. Clinical information is not released without written permission of the subject except as necessary for monitoring by the Food and Drug Administration and the IRB.

Study Discontinuation

The study may be discontinued at any time by the FDA, the IRB or Bristol-Myers Squibb (funding source).

Reporting Requirements

All serious adverse events are reported to the FDA with copies provided to the IRB and Bristol-Myers Squibb.

A serious adverse event is one that meets any of the following criteria:

1) Life-threatening or fatal

2) Substantial or permanent disability

3) Requires or prolongs in-patient hospitalization

4) Cancer

5) A congenital anomaly

6) Overdose

Written Report (10-day): Any adverse event which is serious, unexpected and associated with the use of the drug in clinical studies conducted under an IND must be the subject of a written report to the FDA.

Telephone Report (3-day): Any unexpected fatal or immediately life-threatening adverse event associated with use of the drug in clinical studies conducted under an IND must be the subject of a telephone report to the FDA.

Life-Threatening means that the patient was, in the view of the investigator, at immediate risk of death from the reaction as it occurred.

Unexpected means that the event is not described in the Investigator Brochure and/or the U.S. product labeling regarding the nature, frequency or severity of the event.

Results

The first patient (#28) is a 28 year old white homosexual. He has been infected with HIV-1 for approximately 5 years. In the 3^{rd} year of infection he began HAART (3TC (Epivir)/ D4T (Zerit)/ Efavirenz (Sustiva)). His initial plasma viral RNA was 18,000 copies/ml and his CD4 counts were approximately 500 cells/mm³. Six weeks after the start of therapy his plasma HIV-1 RNA was less than 400 copies/ml (measured by standard Roche RT-PCR and/or branced chain DNA assays, supra), and remained at this level for 28 months prior to entering the study. The HIV-1 plasma RNA level was less than 50 copies/ml as determined by ultrasensitive Roche RT-PCR (supra).

Discussion

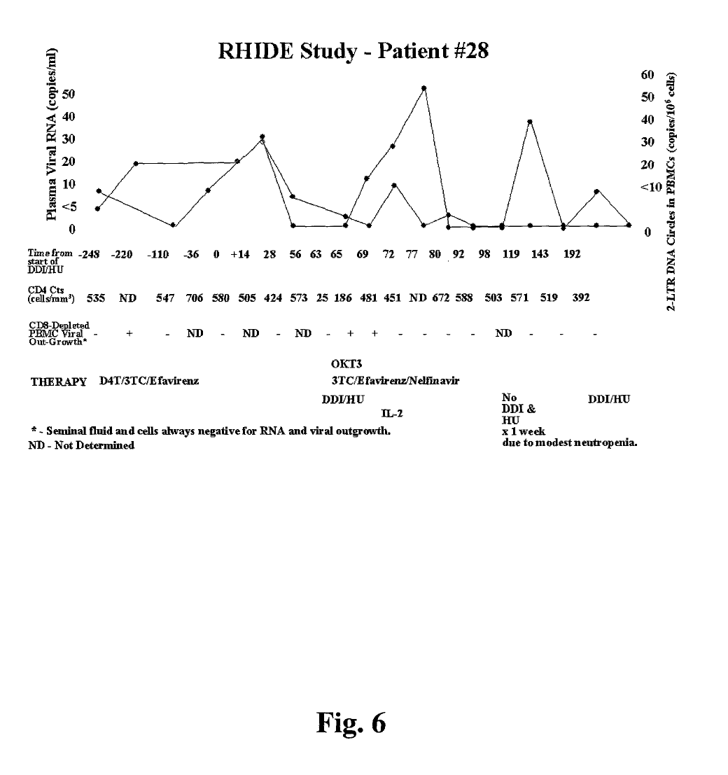

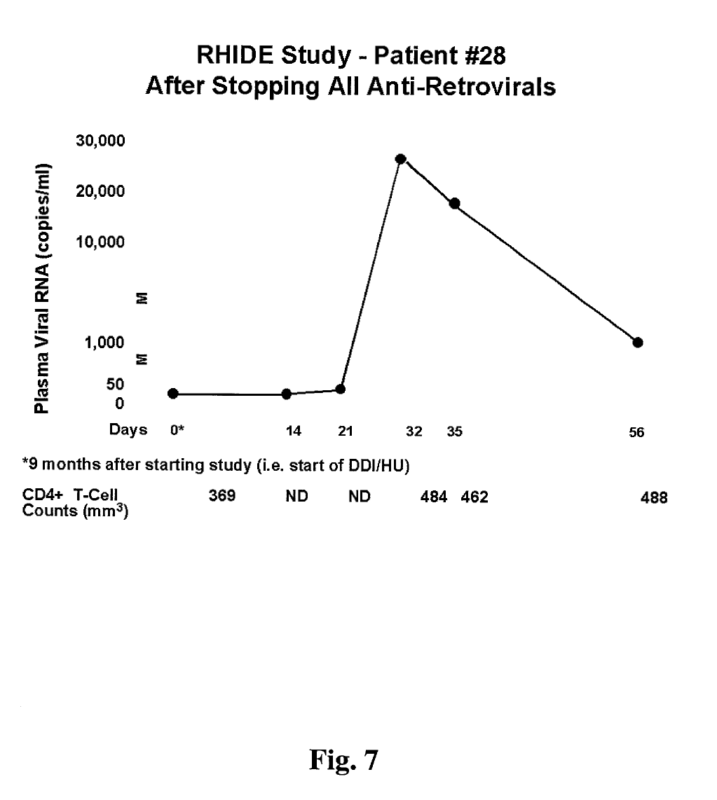

Since the inception of the protocol, two patients have been treated fully and a third is undergoing therapy in this trial. The patient #28 (FIGS. 6 and 7) demonstrated that the patient performed well during the entire trial and did not have serious adverse side-effects from ddI, hydroxyurea, OKT3, or IL-2. Viral replication, as determined by viral out-growth and viral RNA levels, became completely undetectable in the patient using the protocol of the present invention. Tonsillar biopsy analyzed by in situ hybridization and viral out-growth showed no evidence of viral replication in vitro or in vivo, with no viral RNA noted on lymphocytes or follicular dendritic cells. Based on these findings, the patient had all anti-retrovirals stopped nine months after starting ddI and hydroxurea. His plasma viral RNA remained undetectable for three weeks, at which time, on the next RNA analysis at Day 32 after stopping HAART, he had a rebound of viral replication (FIG. 7). As such, this did not demonstrate a complete eradication of HIV1 in this particular patient, but the subsequent course was very promising. Viral replication after the rebound decreased remarkably to approximately 1,000 copies/ml of plasma viral RNA (FIG. 7). This was remarkably lower than the patient's set-point prior to therapy. Thus, there appears to be an immunological effect on viral replication after receiving the residual HIV-1 protocol drugs. The patient is continuing to be followed.

A second patient has also been treated with OKT3 and IL-2 as well as ddI and hydroxurea . This patient also did well during therapy with no serious adverse side-effects. He currently has undetectable viral RNA in his plasma and no viral out-growth. He is scheduled for a tonsillar biopsy and probable stopping of all anti-retrovirals in the next two months.

In summary, the first series of patients have demonstrated that the protocol of the present invention is a useful treatment protocol for HIV-1-infected-individuals on HAART with undetectable virus safely and effectively. Further studies on evaluating both eradication and HIV-1 “remissions” with a low subsequent viral set-point are underway.

What is claimed is:

1. A method for treating a patient with HIV-1, said patient receiving HAART, comprising:
a) selecting said patient for therapy;
b) administering an intensification therapeutic(s), said therapeutic(s) administered in an amount sufficient to block reverse transcriptase;
c) monitoring said patient cells and plasma for decreases in proviral sequences and replication-competent viruses;
d) administering a compound to activate latently-infected cells, said compound administered in an amount sufficient to activate expression of a latent virus;
e) administering a second compound to further activate said latently infected cells, said second compound administered in an amount sufficient to further activate expression of a latent virus;
f) analyzing said patient cells and plasma for said proviral sequences and replication-competent viruses; and
g) eradicating HIV-1.

2. The method of claim 1, wherein said intensification therapeutic(s) comprises hydroxyurea and ddI.

3. The method of claim 1, wherein said compound to activate latently infected cells comprises OKT3.

4. The method of claim 1, wherein said second compound to further activate latently infected cells comprises IL-2.

5. The method of claim 1, wherein activation of said expression of said latent virus comprises depleting proviral resevoirs.

6. The method of claim 1, wherein said cells are within a sanctuary site.

7. The method of claim 6, wherein said sanctuary site is at least one of a gential tract, a central nervous system, or a retina.


SEQUENCE LISTING (3 sequences)

SEQ ID NO: 1 - DNA, 28 bp, Artificial Sequence
  primers for HIV-1
tttggtccttgtcttatgtccagaatgc

SEQ ID NO: 2 - DNA, 28 bp, Artificial Sequence
  primers for HIV-1
ataatccacctatcccagtaggagaaat

SEQ ID NO: 3 - DNA, 41 bp, Artificial Sequence
  probe for HIV-1
atcctgggattaaataaaatagtaagaatgtatagccctac